(19) United States
(12) Patent Application Publication
(10) Pub. No.: US 2003/0082647 A1
(43) Pub. Date: May 1, 2003

(54) Transporter protein

(76) Inventors: Robert A. Reenan, West Hartford, CT (US); Blanka Rogina, West Hartford, CT (US); Stephen L. Helfand, Orange, CT (US)

Correspondence Address: FISH & RICHARDSON PC, 225 FRANKLIN ST, BOSTON, MA, 02110, US

(21) Appl. No.: 10/167,994

(22) Filed: Jun. 12, 2002

Related U.S. Application Data
(63) Continuation-in-part of application No. 10/017,479, filed on Dec. 12, 2001.
(60) Provisional application No. 60/255,013, filed on Dec. 12, 2000.

(51) Int. Cl. G01N 33/567; A61K 49/00

(52) U.S. Cl. 435/7.21; 424/9.2

(57) ABSTRACT

This disclosure provides in part a method of evaluating a compound. The method includes: contacting a carboxylate transporter polypeptide with a test compound; evaluating an interaction between the test compound and the carboxylate transporter polypeptide; contacting a cell or organism that produces the carboxylate transporter polypeptide with the test compound; and evaluating the effect of the test compound on the rate of aging of the cell or organism.

### RELATED APPLICATIONS

This application is a continuation in part of and claims priority to and benefit of each of the following prior applications: U.S. Ser. No. 10/017,479, filed Dec. 12, 2001, and U.S. Ser. No. 60/255,013, filed Dec. 12, 2000, both of which are incorporated herein in their entirety by reference for all purposes.

### STATEMENT REGARDING FEDERALLY SPONSORED RESEARCH

The U.S. Government has certain rights in this invention pursuant to Grant Nos. AG 14532, AG16667 awarded by the National Institute of Health, and 9728737 awarded by the National Science Foundation.

### BACKGROUND

The genetic analysis of aging in model organisms such as C. elegans, Drosophila melanogaster, yeast, and mice has revealed that certain single gene mutations can extend the lifespan of an organism. Thus, it has become well established that the lifespan of metazoans is genetically programmed. Discrete biochemical pathways operate to determine the rate of aging of an organism and, baring disease or accident, approximately when the organism will die. Guarente and Kenyon (2000), Nature 408:255-62.

Similarly, studies of the aging process have indicated that it is controlled by environmental factors as well. In particular, caloric restriction, which typically refers to a diet in which caloric intake is limited to about 30% to 40% of the calories that an animal fed ad libitum would consume, has been shown to extend lifespan in rodents, worms and yeast. See, e.g., Weindruch et al. (1986), Journal of Nutrition 116:641-54. One possibility is that caloric restriction brings about a reduction in metabolism, which in turn slows down the production of toxic oxygen radicals that result in oxidative stress. Oxidative damage to cells has been correlated with aging, although a causal link has not been established.

Given the interest of contemporary society in health and longevity, it is desirable to understand the genetic processes that determine life span and identify the molecules that function to limit or extend life span. By doing so, it will be possible to develop pharmaceutical agents capable of modulating the function of such molecules, thereby allowing the rate of aging to be delayed and life span to be extended.

### SUMMARY OF THE INVENTION

The present invention is based, in part, on the discovery and cloning of a gene, the Drosophila Indy gene, which is involved in the regulation of the rate of aging in Drosophila melanogaster. Flies that are heterozygous for a loss of function mutation in one copy of the Indy gene age at a slower rate than wild-type flies, thus resulting in the extension of their life-span. The Drosophila Indy gene encodes a cation-independent transporter of carboxylates, including Krebs Cycle intermediates such as succinate and citrate. As Drosophila Indy is expressed at the cell surface of cells that line the gut of flies, Drosophila Indy may regulate the rate of aging by controlling caloric intake. The discovery of Drosophila Indy provides cellular targets, e.g., Drosophila Indy or homologs thereof, that can be used to design compounds that modulate the activity of transporter polypeptides, e.g., a transporter of carboxylates, and thereby regulate the rate of aging in cells or organisms.

A “carboxylate transporter polypeptide” refers to a protein that can transport a carboxylate, such as a dicarboxylate (e.g., a dicarboxylic acid) or a tricarboxylates (e.g., a tricarboxylic acid). A carboxylate transporter polypeptide may also transport other substrates. The Drosophila Indy protein is an exemplary carboxylate transporter polypeptide. In addition, this description also includes methods of screening and modulating the activity of other transporter proteins, e.g., a transporter of a sulfate or a metabolite (e.g., a metabolite other than a carboxylate). In one embodiment, the transporter polypeptide is at least 25%, 35%, 45%, 55%, 65%, 75%, 85%, or 95% identical to one or more of the following sequences: SEQ ID NO:2, 3, 4, 5, 6, or 7. In one embodiment, the transporter polypeptide has at least eleven transmembrane domains, e.g., eleven or twelve transmembrane domains. Descriptions below relating to carboxylate transporter polypeptides can also be applied to other transporter polypeptides, e.g., a sodium/sulfate symporter.

Accordingly, in one aspect, the invention features an isolated polynucleotide selected from the group consisting of SEQ ID NO: 1, SEQ ID NO: 1 wherein T can also be U, and nucleic acid sequences substantially complementary to SEQ ID NO: 1.

In a related embodiment, the invention features an isolated polynucleotide encoding an amino acid sequence as set forth in SEQ ID NO:2, or variants of SEQ ID NO:2 comprising conservative amino acid substitutions of SEQ ID NO:2, wherein the variants retain the ability to function as cellular transporters of carboxylates. The invention further encompasses an isolated polynucleotide encoding a polypeptide having greater than or equal to 25% overall identity or greater than or equal to 30% overall similarity to SEQ ID NO:2, wherein the polypeptide is a cellular transporter of carboxylates.

In another aspect, the invention features a polypeptide having the sequence set forth in SEQ ID NO: 2, as well as variants of SEQ ID NO:2 comprising conservative amino acid substitutions of SEQ ID NO:2, and polypeptide having greater than or equal to 25% overall identity or greater than or equal to 30% overall similarity to SEQ ID NO:2.

In another aspect, the invention features methods for isolating an Indy gene comprising contacting a genomic library with one or more DNA probes under conditions effective to produce DNA or RNA copies of the Indy gene; producing copies of the Indy gene; identifying the copies; and isolating the copies; wherein the DNA probe comprises at least 14 contiguous nucleotides of SEQ ID NO: 1.

In another aspect, the invention features an expression vector comprising a polynucleotide selected from the group consisting of SEQ ID NO: 1, SEQ ID NO: 1 wherein T can also be U, and nucleic acid sequences fully complementary to SEQ ID NO: 1, wherein the polynucleotide is operably linked to control sequences that direct transcription of the polynucleotide.

In another aspect, the invention features methods of producing an INDY polypeptide comprising transforming a host cell with an expression vector comprising control sequences that direct transcription of the Indy polynucleotide; expressing the polynucleotide in a host cell; and optionally recovering the INDY polypeptide.

In another aspect, the invention features a host cell, e.g., an oocyte, e.g., a Xenopus oocyte, that produces a heterologous carboxylate transporter, e.g., the Drosophila Indy protein or another transporter described herein. In one embodiment, the transporter includes between one and ten substitutions, deletions, or insertions relative to an amino acid sequence described herein.

In another aspect, the invention features a transgenic organism that contains a transgene encoding a carboxylate transporter polypeptide. In preferred embodiments, the carboxylate transporter polypeptide transports Krebs Cycle intermediates, e.g., succinate or citrate. In other preferred embodiments, the carboxylate transporter polypeptide functions in a cation-independent manner. In other preferred embodiments, the carboxylate transporter polypeptide is a human carboxylate transporter polypeptide. In still other preferred embodiments, the carboxylate transporter polypeptide is not normally found in (i.e., is exogenous to) the transgenic organism. In some embodiments, the transgenic organism is a yeast cell or an insect, e.g., a nematode or a fly, e.g., a Drosophila. In other embodiments, the transgenic organism is a mammal, e.g., a rodent, e.g., a mouse. In still other embodiments, the transgenic organism further comprises a genetic alteration, e.g., a point mutation, insertion, or deficiency, in a gene encoding an endogenous carboxylate transport protein, such that the expression or activity of the endogenous carboxylate transport protein is reduced or eliminated.

In another aspect, the invention features a method to assess the inhibitory activity of a test substance on a polypeptide having greater than or equal to 25% overall identity or greater than or equal to 30% overall similarity to SEQ ID NO:2, comprising contacting the cell with the test substance; and detecting the amount of carboxylate transported by the polypeptide in the presence and absence of the test substance, wherein inhibition of transport in the presence as compared to the absence of the test substance indicates that the test substance is a cellular transporter inhibitor.

In a related aspect, the invention features a method of evaluating a compound, the method comprising: contacting a transporter polypeptide (e.g., a carboxylate transporter polypeptide) with a test compound; evaluating an interaction between the test compound and the transporter polypeptide; contacting a cell or organism that produces the transporter polypeptide with the test compound; and evaluating the effect of the test compound on the rate of aging on the cell or organism. The interaction can, for example, be a physical interaction, e.g., a direct binding interaction, a covalent change in one or both of the test compound or the transporter, a change in location of the test compound (e.g., transport across a lipid bilayer), or a functional interaction (e.g., an alteration in activity, stability, structure, or transport activity of the transporter polypeptide).

In some embodiments, the method is repeated one or more times such that, e.g., a library of test compounds can be evaluated. In an related embodiment, the evaluating of the interaction between the test compound and the transporter polypeptide is repeated, and the evaluating of the rate of aging is selectively used for compounds for which an interaction is detected. Possible test compounds include, e.g., small organic molecules, peptides, antibodies, and nucleic acid molecules.

In preferred embodiments, the transporter polypeptide has greater than or equal to 25% overall identity or greater than or equal to 30% overall similarity to SEQ ID NO:2, 3, 4, 5, 6, or 7. In some embodiments, the carboxylate transporter polypeptide is cation independent. In preferred embodiments, the carboxylate transporter polypeptide is a human protein.

In some embodiments, the interaction between the test compound and the transporter polypeptide is evaluated in vitro, e.g., using an isolated transporter polypeptide. The transporter polypeptide can be in solution (e.g., in a micelle) or bound to a solid support, e.g., a column, agarose beads, a plastic well or dish, or a chip (e.g., a microarray). Similarly, the test compound can be in solution or bound to a solid support.

In other embodiments, the interaction between the test compound and the transporter is evaluated using a cell-based assay. For example, the cell can be a yeast cell, an invertebrate cell (e.g., a fly cell), or a vertebrate cell (e.g., a Xenopus oocyte or a mammalian cell, e.g., a mouse or human cell). In preferred embodiments, the cell-based assay measures the transport activity of the carboxylate transporter polypeptide. For example, the cell-based assay can measure the transport of a carboxylate, e.g., succinate or citrate.

In preferred embodiments, the effect of the test compound on the rate of aging of a cell or animal is evaluated only if an interaction between the test compound and the carboxylate transporter polypeptide is observed.

In some embodiments, the rate of aging of a cell, e.g., a yeast cell, invertebrate cell (e.g., fly cell), or vertebrate cell (e.g., mammalian cell, e.g., human or mouse cell) is determined. For example, the rate of aging of the cell can be evaluated by measuring one or more age-related parameter. Examples of age-related parameters include: the expression of one or more genes or proteins (e.g., genes or proteins that have an age-related expression pattern); resistance to oxidative stress; metabolic parameters (e.g., protein synthesis or degradation, ubiquinone biosynthesis, cholesterol biosynthesis, ATP levels within the cell, glucose metabolism, nucleic acid metabolism, ribosomal translation rates, etc.); cellular proliferation; and physical appearance or behavior. In one embodiment, evaluating the rate of aging includes detecting apoptosis or a sign of apoptosis. A variety of methods are available for assays and signs of apoptosis (e.g., nuclear fragmentation).

In some embodiments, the cell is a transgenic cell. For example, the transgenic cell can include a transgene that encodes a copy of a carboxylate transport protein, e.g., the carboxylate transporter polypeptide that was evaluated for an interaction with the test compound. In some embodiments, the transgene encodes a protein that is normally exogenous to the transgenic cell. In some embodiments, the transgene encodes a human protein, e.g., a human transporter polypeptide, e.g., a human carboxylate transporter polypeptide. In some embodiments, the transgene is linked to a heterologous promoter. In other embodiments, the transgene is linked to its native promoter. In some embodiments, the cell is isolated from an organism that has been contacted with the test compound. In other embodiments, the cell is contacted directly with the test compound.

In other embodiments, the rate of aging of an organism, e.g., an invertebrate (e.g., a worm or a fly) or a vertebrate (e.g., a rodent, e.g., a mouse) is determined. The rate of aging of an organism can be determined by a variety of methods, e.g., by one or more of: a) assessing the life span of the cell or the organism; (b) assessing the presence or abundance of a gene transcript or gene product in the cell or organism that has a biological age-dependent expression pattern; (c) evaluating resistance of the cell or organism to stress, e.g., genotoxic stress (e.g., etopicide, UV irradition, exposure to a mutagen, and so forth) or oxidative stress; (d) evaluating one or more metabolic parameters of the cell or organism; (e) evaluating the proliferative capacity of the cell or a set of cells present in the organism; and (f) evaluating physical appearance or behavior of the cell or organism. In one example, evaluating the rate of aging includes directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined by measuring an age-related parameter. Examples of age-related parameters include: appearance, e.g., visible signs of age; the expression of one or more genes or proteins (e.g., genes or proteins that have an age-related expression pattern); resistance to oxidative stress; metabolic parameters (e.g., protein synthesis or degradation, ubiquinone biosynthesis, cholesterol biosynthesis, ATP levels, glucose metabolism, nucleic acid metabolism, ribosomal translation rates, etc.); and cellular proliferation (e.g., of retinal cells, bone cells, white blood cells, etc.). In some embodiments, the organism is a transgenic animal. The transgenic animal can include a transgene that encodes, e.g., a copy of a carboxylate transport protein, e.g., the carboxylate transporter polypeptide that was evaluated for an interaction with the test compound. In some embodiments, the transgene encodes a protein that is normally exogenous to the transgenic animal. For example, the transgene can encode a human protein, e.g., a human carboxylate transporter polypeptide. In some embodiments, the transgene is linked to a heterologous promoter. In other embodiments, the transgene is linked to its native promoter. In some embodiments, the transgenic animal further comprises a genetic alteration, e.g., a point mutation, insertion, or deficiency, in a gene encoding an endogenous carboxylate transport protein, such that the expression or activity of the endogenous carboxylate transport protein is reduced or eliminated.

In some embodiments, the organism is on a calorically rich diet, while in other embodiments the organism is on a calorically restricted diet.

In some embodiments, a portion of the organism's life, e.g., at least 20%, 30%, 40%, 50%, 60%, 70%, 80%, 90%, or more, of the expected life span of the organism, has elapsed prior to the organism being contacted with the test compound.

In another aspect, the invention features a method of evaluating a protein, comprising: identifying or selecting a candidate protein, wherein the candidate protein is a carboxylate transporter polypeptide; altering the expression or activity of the candidate protein in a cell or in one or more cells of an organism; and determining whether the alteration has an effect on the rate of aging of the cell or organism.

In some embodiments, the candidate protein is identified by amplification of the gene or a portion thereof encoding the candidate protein, e.g., using a method described herein, e.g., PCR amplification or the screening of a nucleic acid library. In preferred embodiments, the candidate protein is identified by searching a database, e.g., searching a sequence database for protein sequences homologous to known carboxylate transporter polypeptides, e.g., Drosophila Indy.

In preferred embodiments, the candidate protein is a human protein. In other embodiments, the candidate protein is a mammalian protein, e.g., a mouse protein. In other embodiments, the protein is a vertebrate protein, e.g., a fish, bird or reptile protein, or an invertebrate protein, e.g., a worm or insect protein. In still other embodiments, the protein is a eukaryotic protein, e.g., yeast protein.

In some embodiments, the cell is an invertebrate cell, e.g., a worm cell or a fly cell. In other embodiments, the cell is a vertebrate cell, e.g., a fish cell (e.g., zebrafish cell), a bird cell (e.g., chicken cell), a reptile cell (e.g., amphibian cell, e.g., Xenopus cell), or a mammalian cell (e.g., mouse or human cell).

In some embodiments, the organism is an invertebrate, e.g., a worm or a fly. In other embodiments, the animal is a non-human vertebrate, e.g., a fish (e.g., zebrafish), a bird (e.g., chicken), a reptile (e.g., amphibian, e.g., Xenopus), or a mammal (e.g., rodent, e.g., mouse).

In preferred embodiments, the expression of the candidate protein is altered so as to reduce the amount of protein, e.g., active protein, present in the cell. For example, the expression of the candidate protein can be reduced by introducing an insertion or deletion into the gene that encodes the protein. Alternatively, the expression of the candidate protein can be reduced by introducing antisense RNA, a ribozyme, or inhibitory double-stranded RNA into the cell. In preferred embodiments, the expression of the candidate protein is reduced by introducing an insertion, deletion, or inhibitory double-stranded RNA into the cell.

In other embodiments, the expression of the candidate protein is altered so as to increase the amount of protein, e.g., active protein, present in the cell. In preferred embodiments, the expression of the candidate protein is increased by introducing a transgene, e.g., plasmid or a viral or transposable element that includes sequence encoding the candidate protein, into the cell or animal.

In some embodiments, the rate of aging of a cell, e.g., a yeast cell, invertebrate cell (e.g., fly cell), or vertebrate cell (e.g., mammalian cell, e.g., human or mouse cell) is determined. For example, the rate of aging of the cell can be evaluated by measuring the expression of one or more genes or proteins (e.g., genes or proteins that have an age-related expression pattern), by measuring the cell's resistance to stress, e.g., genotoxic stress or oxidative stress, by measuring one or more metabolic parameters (e.g., protein synthesis or degradation, ubiquinone biosynthesis, cholesterol biosynthesis, ATP levels within the cell, glucose metabolism, nucleic acid metabolism, ribosomal translation rates, etc.), by measuring cellular proliferation, or any combination of measurements thereof.

In other embodiments, the rate of aging of an organism, e.g., an invertebrate (e.g., a worm or a fly) or a vertebrate (e.g., a rodent, e.g., a mouse) is determined. The rate of aging of an organism can be determined by directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined visually, e.g., by looking for visible signs of age, by measuring the expression of one or more genes or proteins (e.g., genes or proteins that have an age-related expression pattern), by measuring the cell's resistance to oxidative stress, by measuring one or more metabolic parameters (e.g., protein synthesis or degradation, ubiquinone biosynthesis, cholesterol biosynthesis, ATP levels, glucose metabolism, nucleic acid metabolism, ribosomal translation rates, etc.), by measuring cellular proliferation (e.g., of retinal cells, bone cells, white blood cells, etc.), or any combination of measurements thereof. In one embodiment, the visual assessment is for evidence of apoptosis, e.g., nuclear fragmentation.

In another aspect, method of evaluating a protein, the method comprising a) identifying or selecting a candidate protein, wherein the candidate protein is a carboxylate transporter polypeptide; b) identifying one or more polymorphisms in a gene that encodes the candidate protein; and c) assessing correspondence between the presence of one or more of the polymorphisms and the longevity of the organism that contains the polymorphism. The polymorphisms can be naturally occurring or laboratory induced. In one embodiment, the organism is an invertebrate, e.g., a fly or nematode; in another embodiment the organism is a mammal, e.g., a rodent or human. A variety of statistical and genetic methods can be used to assess correspondence between a polymorphism and longevity. Such correlative methods include determination of linkage disequilibrium, LOD scores, and the like.

In another aspect, the invention features a method of decreasing the concentration of a polypeptide having greater than or equal to 25% overall identity or greater than or equal to 30% overall similarity to SEQ ID NO:2 in a cell or extract, comprising contacting the cell or extract with a first nucleic acid molecule in an amount effective to inhibit the expression of a second nucleic acid molecule expressing a cellular transporter of carboxylates, wherein the first nucleic acid molecule is substantially complementary to at least a portion of the Indy gene, and may be an antisense oligonucleotide, a ribozyme, a triple helix-forming molecule, a double stranded interfering RNA, or a mixture comprising at least one of the foregoing.

In another aspect, the invention features a method of altering the expression or activity of a carboxylate transporter polypeptide, comprising administering to a cell or an organism a compound that increases or decreases the expression or activity of the carboxylate transporter polypeptide in an amount effective to increase or decrease the activity of the carboxylate transporter polypeptide.

In some embodiments, the carboxylate transporter polypeptide transports metabolic intermediates, e.g., Krebs Cycle intermediates, e.g., succinate, citrate, or α-keto-glutarate. In some embodiments, the carboxylate transporter polypeptide is a cation-independent carboxylate transporter polypeptide. In preferred embodiments, the transporter polypeptide is a human protein. In other embodiments, the carboxylate transporter polypeptide is a yeast, invertebrate (e.g., worm or fly), or vertebrate (e.g., fish, reptile, bird, or mammal (e.g., mouse)) protein.

In some embodiments, the cell is an invertebrate cell, e.g., a worm cell or a fly cell. In other embodiments, the cell is a vertebrate cell, e.g., a fish cell (e.g., zebrafish cell), a bird cell (e.g., chicken cell), a reptile cell (e.g., amphibian cell, e.g., Xenopus cell), or a mammalian cell (e.g., mouse or human cell).

In some embodiments, the organism is an invertebrate, e.g., a worm or a fly. In other embodiments, the organism is a vertebrate, e.g., a fish (e.g., zebrafish), a bird (e.g., chicken), a reptile (e.g., amphibian, e.g., Xenopus), or a mammal (e.g., rodent or a human). In preferred embodiments, the organism is a human that is not obese or diabetic.

In some embodiments, the compound is an agonist that increases the expression or activity of the carboxylate transporter polypeptide, while in other embodiments the compound is an antagonist that decreases the expression or activity of the carboxylate transporter polypeptide. In preferred embodiments, the agonist is a small organic compound, an antibody, a polypeptide, or a nucleic acid molecule. In preferred embodiments, the antagonist is a small organic compound, an antibody, a polypeptide, or a nucleic acid molecule.

In preferred embodiments, the agonist or antagonist alters the concentration of metabolites, e.g., Krebs Cycle intermediates, e.g., succinate, citrate, or α-keto-glutarate, within the cell or within one or more cells of the organism. In other preferred embodiments, the agonist or antagonist alters the cell's or the organism's resistance to oxidative stress. For example, the antagonist increases the cell's or the organism's resistance to oxidative stress. In still other preferred embodiments, the agonist or antagonist alters one or more aging-related parameters, e.g., the expression of one or more genes or proteins (e.g., genes or proteins that have an age-related expression pattern), or the value of one or more metabolic parameters (e.g., one or more metabolic parameters that reflect the rate of aging of the cell or organism). In particularly preferred embodiments, the agonist or antagonist alters the rate of aging of the cell or organism.

In some embodiments, the compound reduces, e.g., partially reduces, the expression of the carboxylate transporter polypeptide. For example, double-stranded inhibitory RNA, anti-sense RNA, or ribozymes can be used to reduce the expression of the carboxylate transporter polypeptide. In a preferred embodiment, double-stranded inhibitory RNA is used to selectively reduce the expression of one allele of the carboxylate transporter polypeptide, thereby achieving an approximate 50% reduction in the expression of the carboxylate transporter polypeptide.

In another aspect, the invention features a method of altering the rate of aging of a cell or organism. The method includes altering the expression or activity of a carboxylate transporter polypeptide, e.g., in a gut cell of the organism. The method can include other features described herein.

In another aspect, the invention features a method that includes: contacting a Drosophila cell or organism with a test compound, and evaluating the abundance of an mRNA or protein produced from the wingless (wg) or engrailed (en) genes. The evaluating can include evaluating a cell within the antennae. The evaluating can also include comparing the abundance to a reference, e.g., to the abundance of the wg or en mRNA or protein in an untreated or control cell or organism. The method can include the use of a reporter gene or protein, e.g., to measure the abundance of wg or en mRNA or protein.

In a related aspect, the invention features a method of calorically restricting an organism, comprising administering to an organism an antagonist of the activity of a cellular transporter, e.g. a transporter of carboxylates or sulfates, in an amount effective to inhibit the activity of the cellular transporter.

In another related aspect, the invention features a method of treating an organism, comprising administering to an organism an antagonist of the activity of a cellular transporter, e.g. a transporter of carboxylates or sulfates, in an amount effective to inhibit the activity of the cellular transporter. The method can include identifying an organism for an anti-aging treatment, e.g., an organism in need of an anti-aging treatment. The compound can be a small organic molecule, a peptide, an antibody (e.g., an antibody that binds to an extracellular epitope of the transporter), or a nucleic acid. In one embodiment, the rate of aging of the organism is altered. In one embodiment, the organism is a mammal, e.g., a human, e.g., a non-obese or non-diabetic mammal/human.

In yet another aspect, the invention includes methods of treating an organism, comprising administering to an organism a vector encoding SEQ ID NO:1 or an active fragment thereof in an amount effective to increase the body weight of an organism.

The phrase “functional effects” in the context of assays for testing compounds that modulate activity of a transporter protein includes the determination of a parameter that is indirectly or directly under the influence of the transport protein, e.g., a functional, physical, or chemical effect, such as the ability to increase or decrease lifespan or the abundance of a transporter substrate in a cell. “Functional effects” include in vitro, in vivo, and ex vivo activities.

By “determining the functional effect” or “evaluating the functional effect” is meant assaying for a compound that increases or decreases a parameter that is indirectly or directly under the influence of a transporter protein, e.g., a carboxylate transporter, e.g., functional, physical and chemical effects. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g. binding to antibodies; measuring changes in ligand binding activity; measuring cellular proliferation or lifespan; measuring cell surface marker expression; measurement of changes in protein levels for associated sequences; measurement of RNA stability; phosphorylation or dephosphorylation; signal transduction, e.g., receptor-ligand interactions, second messenger concentrations (e.g., cAMP, IP3, or intracellular Ca²⁺); identification of downstream or reporter gene expression (CAT, luciferase, β-gal, GFP and the like), e.g., via chemiluminescence, fluorescence, calorimetric reactions, antibody binding, inducible markers, and ligand binding assays.

“Inhibitors”, “activators”, and “modulators” of a transporter include activating, inhibitory, or modulating molecules identified using in vitro and in vivo assays of transporter proteins. Inhibitors are compounds that, e.g., bind to, partially or totally block activity, decrease, prevent, delay activation, inactivate, or down regulate the activity or expression of a transporter, e.g., antagonists. “Activators” are compounds that increase, open, activate, facilitate, enhance activation, agonize, or up regulate a transporter. Inhibitors, activators, or modulators also include genetically modified versions of transporter proteins, e.g., versions with altered activity, as well as naturally occurring and synthetic ligands, antagonists, agonists, antibodies, antisense molecules, ribozymes, small chemical molecules and the like. Such assays for inhibitors and activators include, e.g., expressing a transporter protein in vitro, in cells, or cell membranes, applying putative modulator compounds, and then evaluating the functional effects on activity, as described above.

Samples or assays of a transporter that are treated with a potential activator, inhibitor, or modulator can be compared to control samples without the inhibitor, activator, or modulator to examine the extent of inhibition. Control samples (untreated with inhibitors) are assigned a relative protein activity value of 100%. Inhibition of a carboxylate transporter, for example, is achieved when the activity value relative to the control is about 80%, preferably 50%, more preferably 25-0%. Activation of a carboxylate transporter, for example, is achieved when the activity value relative to the control (untreated with activators) is 110%, more preferably 150%, more preferably 200-500% (i.e., two to five fold higher relative to the control), more preferably 1000-3000% higher.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, e.g., C. elegans, most preferably a mammal such as a primate e.g., chimpanzee or human; cow; dog; cat; a rodent, e.g., guinea pig, rat, mouse; or a rabbit.

### BRIEF DESCRIPTION OF THE FIGURES

FIG. 1 shows the nucleotide sequence (SEQ ID NO:1) of Drosophila Indy. The full-length cDNA from the ‘ATG’ initiation codon to the ‘TAG’ stop codon is shown.

FIG. 2 shows the deduced amino acid sequence (SEQ ID NO:2) of the INDY polypeptide.

FIG. 3 shows the genomic organization of the Indy locus with the insertion sites of all five P-element alleles, wherein the black boxes represent conserved regulatory sequences; the gray box represents the conserved Hoppel transposable element; PlacW insertions sites in the 206, 302, and 159 insertion lines are shown, as well as orientation of the insertions; and positions of Birmingham-2 P-element insertions (PBm) in the 92 and 265 insertions lines are also shown.

FIG. 4 shows life-span extension in Indy mutants, wherein survival curves of males heterozygous for three different Indy mutations and an enhancer-trap control are compared. All flies were tested as heterozygotes for the Indy mutant and a wild-type Canton-S chromosome. The Indy mutants are Indy302 (open circle), Indy206 (closed circle), and Indy159 (closed triangle). The control (closed box) is one of four other enhancer-trap control lines from the same mutagenesis that generated Indy302 and Indy206 tested as a heterozygote over a wild-type Canton-S chromosome. A similar control survival curve was found for a control from the mutagenesis that gave rise to Indy159.

FIG. 5 shows the reversion of life-span extension upon P-element removal. Survival curves of males heterozygous for three different Indy mutations, a precise excision of the P-element from Indy 302 (revertant), and an enhancer-trap control are shown. The Indy mutants are Indy302 (open circle), Indy206 (closed circle), and Indy159 (closed triangle). The excision line (revertant-open box) is one of four exact excisions (sequence confirmed) of the P-element obtained by mobilizing the P-element from either the Indy302 or Indy206 line. The control (closed box) is one of four other enhancer-trap control lines from the same mutagenesis that generated Indy302 and Indy206 tested as a heterozygote over a wild-type Canton-S chromosome.

FIG. 6 shows the survival curve for a control hyperkinetic line (closed circles) and a hyperkinetic line crossed with the Indy 206 mutant line (open squares).

FIG. 7 shows the survival curve for the luckinbill Il 6 line (triangle), the luckinbill Il-6 line crossed with the 1085 line as a control (diamond), the luckinbill Il-6 line crossed with the wg line as a control (square) and the luckinbill Il-6 line crossed with Indy line 206 (circle).

FIG. 8 shows the rate of aging for a normal fly (squares) and Indy heterozygous flies (triangles and circles).

FIG. 9. shows the alignment of INDY with homologous proteins. The most homologous proteins to the INDY protein (GenBank accession AF217399) were identified by Blast. Indy-2 is a highly homologous Drosophila gene (AE003728). SDCT1 (AF058714) and SDCT2 (AF081825) are rat sodium dicarboxylate cotransporters, and hNaDC-1 (U26209) is a human dicarboxylate cotransporter. The boxes indicate either identity or similarity to INDY.

FIG. 10 shows a schematic of the structure of a sodium dicarboxylate cotransporter. The model shows 11 transmembrane domains, an intracellular amino terminal domain, and a carboxyl terminal extracellular domain.

FIG. 11 shows the expression of Indy in adult flies. Whole mount X-gal staining shows nuclear localization of β-gal in cells from lines carrying an enhancer-trap insert in the Indy gene—Indy302, Indy206, and Indy159. Expression is seen in oenocytes (A, B) and gut (C, D). Low power views of oenocytes in the (v) ventral and (d) dorsal abdominal segments are shown in (A). A high power view of dorsal midline oenocytes is shown in (B). Panel (D) shows a 5 mm section showing X-gal staining within the cells of the gut. After whole mount X-gal staining, the tissue in (D) was postfixed in 6.25% glutaraldehyde, embedded in paraffin, and then sectioned. The scale bar in A, B, and C is 100 mm. In (D) the scale bar is 10 mm.

FIG. 12 shows the subcellular localization of INDY using an anti-INDY antibody. INDY is localized to the plasma membrane of midgut epithelial cells, fat body cells, and oenocytes (Dark staining in panels A, B, and D. Panel C is an immunofluorescence image of the midgut showing staining expected from basolateral proteins. Dark staining shows antibody localization to the plasma membranes of midgut (dark staining) localized to the basolateral aspects of the midgut.

FIG. 13 shows the uptake of [¹⁴C] succinate in the presence of NaCl by Xenopus oocytes injected with the Indy mRNA or an H₂O control.

FIG. 14 shows the inhibition of [¹⁴C]succinate uptake in Xenopus oocytes expressing the Indy mRNA in the presence of succinate, citrate, alpha-ketoglutarate, fumarate, pyruvate, glutamate, lactate, or sulfate.

FIG. 15 shows the cation independence of succinate uptake in Xenopus oocytes expressing the Indy mRNA. The [¹⁴C]succinate uptake is measured in the presence of NaCl, KCl, LiCl and CholineCl. A control in which oocytes were injected with H₂O shows no succinate uptake.

FIG. 16 shows the pH-independence of [¹⁴C] succinate uptake in Xenopus oocytes injected with H₂O or the Indy mRNA in the presence of NaCl or Na gluconate (NaGluco).

FIG. 17 shows the citrate inhibition of [¹⁴C]succinate uptake in Xenopus oocytes injected with H₂O or the Indy mRNA. Citrate is added at 10 mM, 1 mM and 0.1 mM concentrations.

FIG. 18 shows the effect of ion channel inhibitors on [¹⁴C]succinate uptake in Xenopus oocytes injected with H₂O or the Indy mRNA. The inhibitors used are 10 mM p-aminohippuric Acid (PAH), 1 mM phloretin and 0.1 mM 4,4′-diisothyocyanostilbene-2,2′-disulfonic acid (DIDS).

FIG. 19 shows the egg production of an Indy heterozygous female (open squares) compared to a normal female (closed squares) under high calorie conditions.

FIG. 20 shows the egg production of an Indy heterozygous female (open squares) compared to a normal female (closed squares) under low calorie conditions.

FIG. 21 shows survival curves for a normal fly fed normal calorie food (circles) or low calorie food (squares).

FIG. 22 shows survival curves for an Indy/Indy heterozygote fly fed normal calorie food (circles) or low calorie food (squares).

### DETAILED DESCRIPTION

The present disclosure originates from the discovery and cloning of a gene, the Drosophila Indy gene, which is involved in the regulation of life span in Drosophila melanogaster. As used herein the term “gene” means the segment of DNA involved in producing a polypeptide chain; it includes regions preceding and following the coding region (leader and trailer) as well as intervening sequences (introns) between individual coding segments (exons). The Drosophila Indy gene encodes a polypeptide that has similarity to dicarboxylate transporters such as those from human and rat. Specifically, identification of Drosophila Indy resulted from the observation that particular mutations in the gene cause an increase in the life span of the fly carrying the mutation. As a result of this finding, it is now possible to identify and/or isolate Drosophila lines with longer life spans, as well as to identify agents that contribute to longer life span. It is further possible to isolate the genes involved in and which have an effect on longevity, as well as the proteins encoded by these genes.

The Drosophila Indy gene was identified from studies of Drosophila enhancer-trap lines, when it was observed that certain fly lines (namely lines 206 and 302) had extended life spans. The genomic DNA flanking the site of insertion in the enhancer-trap lines was sequenced. Both insertion sites were in the same gene that was named Indy.

Indy Nucleic Acid Molecules

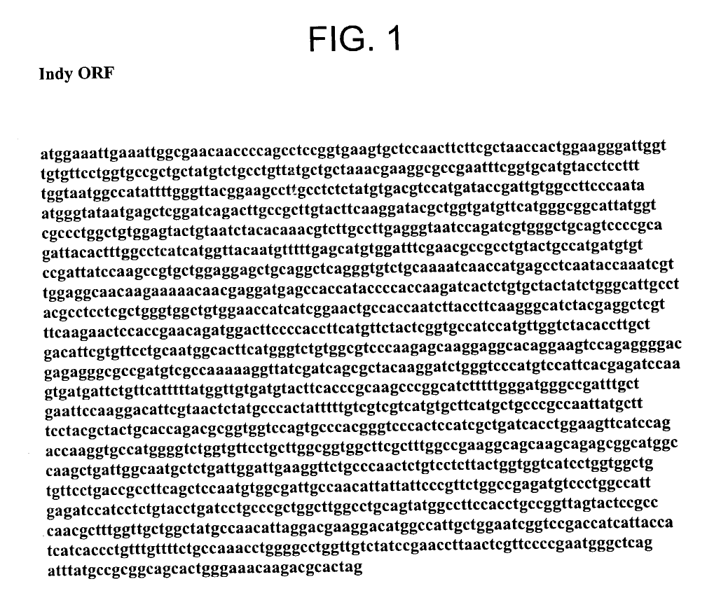

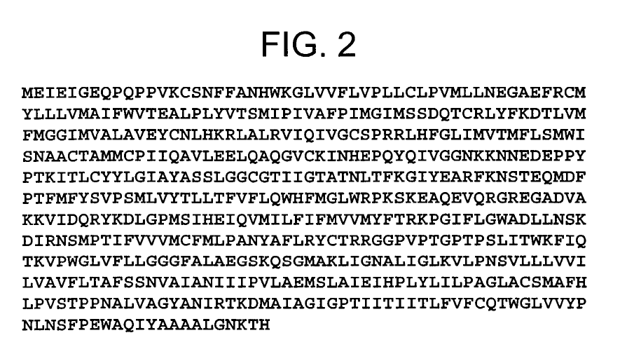

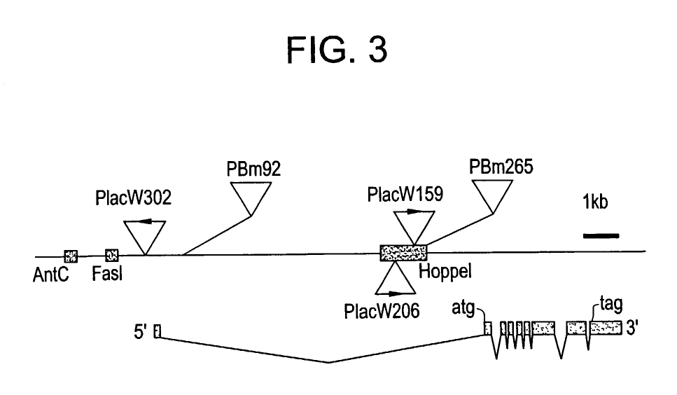

The cDNA sequence and deduced amino acid sequence of Drosophila Indy are shown in FIGS. 1 and 2, respectively. The genomic organization of the Drosophila Indy gene is shown in FIG. 3. A cDNA encoding the open reading frame of Drosophila Indy or portions thereof can be incorporated into commercially available bacterial expression plasmids such as the pGEM (Promega) or pBluescript (Stratagene) vectors or one of their derivatives. When the Drosophila Indy cDNA incorporated into a plasmid is transcribed by an appropriate RNA polymerase, the Drosophila Indy mRNA is produced. Indy mRNA is useful for in vivo and in vitro production of INDY polypeptides.

Accordingly, in one embodiment, this disclosure provides an isolated polynucleotide sequence encoding the Drosophila INDY polypeptide. By “isolated nucleic acid sequence” is meant a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an automatically replicating plasmid or virus; or into the genomic DNA of a prokaryote or eukaryote, or which exists as a separate molecule (e.g., a cDNA) independent of other sequences. “Polynucleotide” or “nucleic acid sequence” refers to a polymeric form of nucleotides at least 5 bases in length. The nucleotides can be ribonucleotides, deoxyribonucleotides, or modified forms of either nucleotide. Modifications include but are not limited to known substitutions of a naturally-occurring base, sugar or internucleoside (backbone) linkage with a modified base such as 5-methylcytosine, a modified sugar such as 2′-methoxy and 2′-fluoro sugars, and modified backbones such as phosphorothioate and methyl phosphonate.

A polynucleotide can be a DNA molecule, a cDNA molecule, genomic DNA molecule, or an RNA molecule. A polynucleotide as DNA or RNA can include a sequence wherein T (thymidine) can also be U (uracil). The polynucleotide can be complementary to SEQ ID NO: 1, wherein complementary refers to the capacity for precise pairing between two nucleotides. For example, if a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process. As used herein, hybridization means Watson-Crick hydrogen bonding between complementary nucleoside or nucleotide bases.

In addition, polynucleotides encoding all or a portion of Indy are included, so long as they encode a polypeptide with INDY activity, such as the transport of carboxylates. Such polynucleotides include naturally occurring, synthetic and intentionally manipulated DNA molecules. For example, the Indy polynucleotide may be subjected to site-directed mutagenesis by techniques known in the molecular biology art. There are 20 naturally occurring amino acids, most of which are specified by more than one codon. Therefore, degenerate nucleotide sequences are included as long as the INDY polypeptide encoded by the nucleotide sequence is functionally unchanged. Also included are polynucleotide sequences that encode amino acid sequences which differ from those of the Indy gene, but which should not produce phenotypic changes.

The Indy polynucleotides also include polynucleotides coding for polypeptide analogs, fragments or derivatives of antigenic polypeptides which differ from naturally-occurring Indy forms in terms of the identity or location of one or more amino acid residues (deletion analogs containing less than all of the residues specified for the polypeptide, substitution analogs wherein one or more residues specified are replaced by other residues and addition analogs where in one or more amino acid residues is added to a terminal or medial portion of the polypeptide) and which share some or all properties of naturally-occurring forms. These molecules include the incorporation of codons suitable for expression by selected non-mammalian hosts; the provision of sites for cleavage by restriction endonuclease enzymes; and the provision of additional initial, terminal or intermediate DNA sequences that facilitate construction of readily expressed vectors.

The Indy polynucleotides include polynucleotides that encode INDY polypeptides or full-length protein that contain substitutions, insertions, or deletions into the protein backbone. Related polypeptides are aligned with INDY by assigning degrees of homology to various deletions, substitutions and other modifications. Homology can be determined along the entire polypeptide or polynucleotide or along subsets of contiguous residues. The percent identity is the percentage of amino acids or nucleotides that are identical when the two sequences are compared. The percent similarity is the percentage of amino acids or nucleotides that are chemically similar when the two sequences are compared. INDY and a homologous polypeptide are preferably greater than or equal to 25%, preferably greater than or equal to 30%, more preferably greater than or equal to 35% or most preferably greater than or equal to 40% identical. INDY and a homologous polypeptide are preferably greater than or equal to 30%, preferably greater than or equal to 35%, more preferably greater than or equal to 45% similar.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Of course, many other polypeptides will meet the same criteria.

The polynucleotide includes SEQ ID NO: 1 as well as complementary sequences to that sequence. When the sequence is RNA, the nucleotide T in SEQ ID NO:1 is U. In addition, polynucleotides that are substantially identical to SEQ ID NO:1 or which encode proteins substantially identical to SEQ ID NO:2 are included. By “substantially identical” is meant a polypeptide or polynucleotide having a sequence that is at least 85%, preferably 90%, and more preferably 95% or more identical to the sequence of the reference amino acid or nucleic acid sequence. For polypeptides, the length of the reference polypeptide sequence will generally be at least 16 amino acids, preferably at least 20 amino acids, more preferably at least 25 amino acids, and most preferably at least 35 amino acids. For nucleic acids, the length of the reference nucleic acid sequence will generally be at least 50 nucleotides, preferably at least 60 nucleotides, more preferably at least 75 nucleotides, and most preferably 110 nucleotides.

Sequence identity and similarity is measured using sequence analysis software (e.g., Sequence Analysis Software Package of the Genetics Computer Group, University of Wisconsin Biotechnology Center, 1710 University Avenue, Madison, Wis. 53705) with the following parameters: Blossum 62 scoring matrix with a gap penalty of 12, a gap extend penalty of 4, and a frameshift gap penalty of 5. The percent identity between the two sequences is a function of the number of identical positions shared by the sequences, taking into account the number of gaps, and the length of each gap, which need to be introduced for optimal alignment of the two sequences.

This disclosure also encompasses DNAs and cDNAs that hybridize to the Indy polynucleotide. Hybridization methods are well known to those of ordinary skill in the art. The hybridizing sequences can be nucleic acid sequences of greater than about 14 nucleotides in length that selectively hybridize to an Indy polynucleotide.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

The Indy polynucleotide can also be designed to provide additional sequences, such as, for example, the addition of coding sequences for added C-terminal or N-terminal amino acids that would facilitate purification by trapping on columns or use of antibodies. Such tags include, for example, histidine-rich tags that allow purification of polypeptides on Nickel columns. Such gene modification techniques and suitable additional sequences are well known in the molecular biology arts.

The Indy polynucleotide can be inserted into a recombinant DNA vector for production of Indy mRNA. Such vectors may be used for the in vitro or in vivo production of Indy mRNA. For in vitro production of Indy mRNA, the cDNA comprising SEQ ID NO:1, for example, is inserted into a plasmid containing a promoter for either SP6 or T7 RNA polymerase. The plasmid is cut with a restriction endonuclease to allow run-off transcription of the mRNA, and the RNA is produced by addition of the appropriate buffer, ribonucleotides, and polymerase. The RNA is isolated by conventional means such as ethanol precipitation. The mRNA can be capped or polyadenylated, for example, prior to injection into a cell such as a Xenopus oocyte.

The Indy polynucleotide can be inserted into a recombinant expression vector. The term “recombinant expression vector” refers to a plasmid, virus, or other means known in the art that has been manipulated by insertion or incorporation of the Indy genetic sequence. The term “plasmids” generally is designated herein by a lower case “p” preceded and/or followed by capital letters and/or numbers, in accordance with standard naming conventions that are familiar to those of skill in the art. Plasmids disclosed herein are either commercially available, publicly available on an unrestricted basis, or can be constructed from available plasmids by routine application of well-known, published procedures. Many plasmids and other cloning and expression vectors are well known and readily available, or those of ordinary skill in the art may readily construct any number of other plasmids suitable for use. These vectors may be transformed into a suitable host cell to form a host cell vector system for the production of a polypeptide having the biological activity of a cellular transporter. Suitable hosts include microbes such as bacteria, yeast, insect or mammalian organisms or cell lines.

The Indy genetic sequence can be inserted into a vector adapted for expression in a bacterial, yeast, insect, amphibian, or mammalian cell that further comprises the regulatory elements necessary for expression of the nucleic acid molecule in the bacterial, yeast, insect, amphibian, or mammalian cell operatively linked to the nucleic acid molecule encoding a cellular transporter of carboxylates as to permit expression thereof. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., “ATG”) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included (see e.g., Bitter et al., Methods in Enzymology 153: 516-544, 1987).

Examples of suitable bacteria are E. coli and B. subtilis. A preferred yeast vector is pRS426-Gal. Examples of suitable yeast are Saccharomyces and Pichia. Suitable amphibian cells are Xenopus cells. Suitable vectors for insect cell lines include baculovirus vectors. Rat or human cells are preferred mammalian cells.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell. By “transformed cell” or “host cell” is meant a cell (e.g., prokaryotic or eukaryotic) into which (or into an ancestor of which) has been introduced, by means of recombinant DNA techniques, a DNA molecule encoding a polypeptide of the invention (i.e., an INDY polypeptide), or fragment thereof.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with DNA sequences encoding a polypeptide of this disclosure, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Preferably, a eukaryotic host is utilized as the host cell as described herein. The eukaryotic cell may be a yeast cell (e.g., Saccharomyces cerevisiae) or may be a mammalian cell, including a human cell.

Mammalian cell systems that utilize recombinant viruses or viral elements to direct expression may be engineered. For example, when using adenovirus expression vectors, the nucleic acid sequences encoding a foreign protein may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the INDY polypeptide in infected hosts (e.g., Logan & Shenk, Proc. Natl. Acad. Sci. U.S.A. 81:3655-3659, 1984).

For long-term, high-yield production of recombinant proteins, stable expression is preferred. Rather than using expression vectors that contain viral origins of replication, host cells can be transformed with the cDNA encoding an INDY fusion protein controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci, which in turn can be cloned and expanded into cell lines. For example, following the introduction of foreign DNA, engineered cells may be allowed to grow for 1 to 2 days in an enriched media, and then are switched to a selective media. A number of selection systems may be used, including but not limited to the herpes simplex virus thymidine kinase (Wigler et al., Cell 11: 233, 1977), hypoxanthine-guanine phosphoribosyltransferase (Szybalska & Szybalski, Proc. Natl. Sci. U.S.A. 48: 2026, 1962), and adenine phosphoribosyltransferase (Lowy et al., Cell 22: 817, 1980) genes can be employed in tk, hgprt or aprt cells respectively. Among the known methods for expressing transporter genes is expression in a Xenopus oocyte system. A cDNA encoding the open reading frame of Indy or portions thereof can be incorporated into commercially available bacterial expression plasmids such as the pGEM (Promega) or pBluescript (Stratagene) vectors or one of their derivatives. After amplifying the expression plasmid in bacterial (E. coli) cells the DNA is purified by standard methods. The incorporated transporter sequences in the plasmid DNA are then transcribed in vitro according to standard protocols, such as transcription with SP6 or T7 RNA polymerase. The RNA thus prepared is injected into Xenopus oocytes where it is translated and the resulting transporter polypeptides are incorporated into the plasma membrane. The functional properties of these transporters can then be investigated by electrophysiological, biochemical, pharmacological, and related methods.

Additional Transporter Polypeptides

In addition to the sequences described above, full-length Indy cDNA, gene sequences or paralogs present in the same species or orthologs of the Indy gene in other species can readily be identified without undue experimentation, by molecular biological techniques well known in the art. The identification of orthologs of Indy can be useful for developing model animal systems more closely related to humans for use in drug design. “Homolog” is a generic term used in the art to indicate a polynucleotide or polypeptide sequence possessing a high degree of sequence relatedness to a subject sequence. Such relatedness may be quantified by determining the degree of identity and/or similarity between the sequences being compared as hereinbefore described. Falling within this generic term are the terms “ortholog”, meaning a polynucleotide or polypeptide that is the functional equivalent of a polynucleotide or polypeptide in another species, and “paralog” meaning a functionally similar sequence when considered within the same species.

Genes that regulate life span or senescence can be isolated by isolation of DNA homologous to other genes known to regulate life span, for example the Indy gene. A gene library from an organism of interest can be probed using protocols well known in the art. The gene library is preferably a mammalian gene library and more preferably a human gene library. Homologous genes can be isolated by hybridization. For example, a labeled DNA fragment comprising the Indy gene is used to probe cellular DNA from an organism of interest under high, medium or low hybridization stringency conditions, depending on the degree of homology sought, for example as taught in Sambrook et al., Eds., Molecular Cloning: A Laboratory Manual, 2nd ed., Cold Spring Harbor Laboratory Press, 1989, or Ausubel, F. M. et al., eds. Current Protocols in Molecular Biology, 1994. DNA hybridizing to the probe is isolated, and complementation analysis is performed to verify that the DNA comprises a gene that contributes to longevity. DNA from an organism of interest can be hybridized under high stringency conditions to DNA comprising a mutated Indy gene. A preferred Indy DNA probe is greater than or equal to 14 contiguous nucleotides of SEQ ID NO:1.

Homologous genes can also be found by the polymerase chain reaction (PCR) (see Sakai et al., Science 230: 1350-4, 1985; and Sakai et al., Science 239: 487-91, 1988). Synthetic oligonucleotide primers that comprise regions of the Indy gene can be used. The term “oligonucleotide” as used herein is defined as a molecule comprising 2 or more deoxyribonucleotides or ribonucleotides, preferably more than 3, and most preferably more than about 10. Further as used herein the term “oligonucleotide” comprises less than about 100, more preferably less than about 80, most preferably less than about 50 deoxyribonucleotides or ribonucleotides. The exact size of an oligonucleotide will depend on many factors, including the ultimate function or use of the oligonucleotide. Oligonucleotides can be prepared by any suitable method, including, for example, cloning and restriction of appropriate sequences and direct chemical synthesis by a method such as the phosphotriester method of Narang et al., Meth. Enzymol. 68: 90-99, 1979; the phosphodiester method of Brown et al., Method Enzymol. 68: 109-151, 1979, the diethylphosphoramidite method of Beaucage et al, Tetrahedron Lett. 22: 1859-1862, 1981; the triester method of Matteucci et al., J. Am. Chem. Soc. 103: 3185-3191, 1981, or automated synthesis methods; and the solid support method of U.S. Pat. No. 4,458,066.

The term “primer” as used herein refers to an oligonucleotide that is capable of acting as a point of initiation of synthesis when placed under conditions in which primer extension is initiated or possible. Synthesis of a primer extension product that is complementary to a nucleic acid strand is initiated in the presence of nucleoside triphosphates and a polymerase in an appropriate buffer at a suitable temperature. The term primer may refer to more than one primer, particularly in the case where there is some ambiguity in the information regarding one or both ends of the target region to be synthesized. For instance, if a nucleic acid sequence is inferred from a protein sequence, a primer generated to synthesize nucleic acid encoding said protein sequence is actually a collection of primer oligonucleotides containing sequences representing all possible codon variations based on the degeneracy of the genetic code. One or more of the primers in this collection will be homologous with the end of the target sequence. Likewise, if a “conversed” region shows significant levels of polymorphism in a population, mixtures of primers can be prepared that will amplify adjacent sequences. For example, primers can be synthesized based upon the amino acid sequence as set forth in SEQ ID NO:2 and can be designed based upon the degeneracy of the genetic code.

In one embodiment, synthetic oligonucleotide primers that comprise the region of the Indy gene that contains a mutation are used. Alternatively, oligonucleotides can be patterned after any gene, such as those isolated by this method or any of the above methods, which regulate senescence or life span. The oligonucleotides are utilized in PCR to generate multiple copies of DNA of interest from a sample of genomic DNA from the organism of interest. The DNA multiplied in PCR is then isolated, and complementation analysis is performed to verify that the DNA comprises a functional gene that regulate senescence or life span. Once genes have been isolated using these methods, standard procedures can then be used to isolate the proteins encoded by the genes.

Homologous genes can also be found by computerized database searches to identify genes that include regions of homology to the Indy or other homologous genes. Sequence analysis software matches the similar sequences by assigning degrees of homology to various deletions, substitutions and other modifications. Homologous or identical sequences have a specified percentage of amino acid residues or nucleotides the same when aligned for maximal correspondence over a specified comparison window. The comparison window can be 20 to 600 nucleotides or amino acids. A useful program is BLAST, which is described in Altschul et al., Nucl. Acids Res. 25: 3389-3402, 1977; and Altschul et al., J. Mol. Biol. 215: 403-410, 1990.

In another embodiment, the invention features a method of evaluating a protein or identifying a target, e.g., a protein target suitable for use in the identification of compounds that ameliorate or delay the effects of aging by reducing the rate of aging, comprising: identifying a candidate protein in a cell or organism, wherein the candidate protein is a carboxylate transporter polypeptide; altering the expression of the candidate protein in the cell or in one or more cells of the organism; and determining whether the alteration has an effect on the rate of aging of the cell or organism.

The candidate protein can be identified by amplification of the gene or a portion thereof encoding the candidate protein, e.g., using a method described herein, e.g., PCR amplification or the screening of a nucleic acid library. In preferred embodiments, the candidate protein is identified by searching a database, e.g., searching a sequence database for protein sequences homologous to known carboxylate transporter polypeptides, e.g., Drosophila Indy.

Preferably, the candidate protein is a human protein. Alternatively, the candidate protein can be a mammalian protein, such as a mouse protein, or a protein from a yeast, worm, fly, fish, reptile, or bird.

The expression of the candidate protein is altered so as to reduce the amount of protein, e.g., active protein, present in the cell or animal. For example, the expression of the candidate protein can be reduced by introducing an insertion or deletion into the gene that encodes the protein. Alternatively, the expression of the candidate protein can be reduced by introducing antisense RNA, a ribozyme, or inhibitory double-stranded RNA into the cell or animal. The expression of the candidate protein can also be altered so as to increase the amount of protein, e.g., active protein, present in the cell or animal. Increasing the expression of the candidate protein can be achieved by introducing a transgene, e.g., plasmid or a viral or transposable element that includes sequence encoding the candidate protein, into the cell or animal.

The rate of aging of the cell or organism can be evaluated by measuring the expression of one or more genes or proteins (e.g., genes or proteins that have an age-related expression pattern), by measuring the cell's resistance to oxidative stress, by measuring one or more metabolic parameters (e.g., protein synthesis or degradation, ubiquinone biosynthesis, cholesterol biosynthesis, ATP levels within the cell, glucose metabolism, nucleic acid metabolism, ribosomal translation rates, etc.), by measuring cellular proliferation (e.g., of retinal cells, bone cells, white blood cells, etc.), or any combination of measurements thereof, as described herein.

In another embodiment, the invention features a method of evaluating a protein or identifying a target, the method comprising: identifying a candidate protein in an organism, wherein the candidate protein is a carboxylate transporter polypeptide; identifying one or more polymorphisms in a gene that encodes the candidate protein; and determining whether there is a correlation between the presence of one or more of the polymorphisms and the longevity of the organism that contains the polymorphism.

Methods for determining whether there is linkage between a polymorphism and a physical trait, such as longevity, are well known in the art. For example, groups of long-lived subjects can be screened for the presence of polymorphism in the gene encoding the candidate protein, and the frequency of the polymorphism in the long-lived set of subjects can be compared to the frequency of a group of randomly selected subjects to see if there is a statistically significant difference.

Based on the phenotype of the Drosophila Indy mutations, a suitable target would one in which the reduction in protein levels within a cell or organism results in an increase in life span. Similarly, the target would be expected to decrease life span when over expressed.

The polynucleotides described and claimed herein are useful for the information that they provide concerning the amino acid sequence of the polypeptide and as products for the large scale synthesis of the polypeptide by a variety of recombinant techniques. The polynucleotides are useful for generating new cloning and expression vectors, transformed and transfected prokaryotic and eukaryotic host cells, and new and useful methods for cultured growth of such host cells capable of expression of the polypeptide and related products.

Genes that are suitable for use in the methods of the invention include human carboxylate transporters (e.g., hNaDC-1, accession No. U26209), rat carboxylate transporters (e.g., SDCT2, accession no. AF081825), rabbit carboxylate transporters (e.g., NaDC-1, accession no. U12186) and mouse carboxylate transporters (e.g., mNaDC-1, accession no. AF 201903).

Additional examples of genes/proteins that are suitable for use in the methods of the present invention include the human solute carrier family 13, member 2, which has the following sequence:

(SEQ ID NO:3; see also Gen Bank NP_003975.1)
MATCWQALWAYRSYLIVFFVPILLLPLPILVPSKEAYCAYAIILMALFWC
TEALPLAVTALFPLILFPMMGIVDASEVAVEYLKDSNLLFFGGLLVAIAV
EHWNLHKRIALRVLLIVGVRPAPLILGFMLVTAFLSMWISNTATSAMMVP
IAHAVLDQLHSSQASSNVEEGSNNPTFELQEPSPQKEVTKLDNGQALPVT
SASSEGRAHLSQKHLHLTQCMSLCVCYSASIGGIATLTGTAPNLVLQGQI
NSLFPQNGNVVNFASWFSFAFPTMVILLLLAWLWLQILFLGFNFRKNFGI
GEKMQEQQQAAYCVIQTEHRLLGPMTFAEKAISILFVILVLLWFTREPGF
FLGWGNLAFPNAKGESMVSDGTVAIFIGIIMFIIPSKFPGLTQDPENPGK
LKAPLGLLDWKTVNQKMPWNIVLLLGGGYALAKGSERSGLSEWLGNKLTP
LQSVPAPAIAIILSLLVATFTECTSNVATTTIFLPILASMAQAICLHPLY
VMLPCTLATSLAFMLPVATPPNAIVFSFGDLKVLDMARAGFLLNIIGVLI
IALAINSWGIPLFSLHSFPSWAQSNTTAQCLPSLANTTTPSP

Another example is the human sodium-dependent high-affinity dicarboxylate transporter 3, which has the following sequence:

(SEQ ID NO:4)
MAALAAAAKKVWSARRLLVLLFTPLALLPVVFALPPKEGRCLFVILLMAV
YWCTEALPLSVTALLPIVLFPFMGILPSNKVCPQYFLDTNFLFLSGLIMA
SAIEEWNLHRRIALKILMLVGVQPARLILGMMVTTSFLSMWLSNTASTAM
MLPIANAILKSLFGQKEVRKDPSQESEENTAAVRRNGYTLCPRRCSFSPS
TEAKDHPGETEVPLDLPADSRKEDEYRRNIWKGFLISIPYSASIGGTATL
TGTAPNLILLGQLKSFFPQCDVVNFGSWFIFAFPLMLLFLLAGWLWISFL
YGGLSFRGWRKNKSEIRTNAEDRARAVIREEYQNLGPIKFAEQAVFILFC
MFAILLFTRDPKFIPGWASLFNPGFLSDAVTGVAIVTILFFFPSQRPSLK
WWFDFKAPNTETEPLLTWKKAQETVPWNIILLLGGGFAMAKGCEESGLSV
WIGGQLHPLENVPPALAVLLITVVIAFFTEFASNTATIIIFLPVLAELAI
RLRVHPLYLMIPGTVGCSFAFMLPVSTPPNSIAFASGHLLVKDMVRTGLL
MNLMGVLLLSLAMNTWAQTIFQLGTFPDWADMYSVNVTALPPTLANDTFR
TL

Another example is the human carrier family 13 (sodium/sulfate symporters), member 1, which has the following sequence:

(SEQ ID NO:5)
MKFFSYILVYRRFLFVVFTVLVLLPLPIVLHTKEAECAYTLFVVATFWLT
EALPLSVTALLPSLMLPMFGIMPSKKVASAYFKDFHLLLIGVICLATSIE
KWNLHKRIALKMVMMVGVNPAWLTLGFMSSTAFLSMWLSNTSTAAMVMPI
AEAVVQQIINAEAEVEATQMTYFNGSTNHGLEIDESVNGHEINERKEKTK
PVPGYNNDTGKISSKVELEKNSGMRTKYRTKKGHVTRKLTCLCIAYSSTI
GGLTTITGTSTNLIFAEYFNTRYPDCRCLNFGSWFTFSFPAALIILLLSW
IWLQWLFLGFNFKEMFKCGKTKTVQQKACAEVIKQEYQKLGPIRYQEIVT
LVLFIIMALLWFSRDPGFVPGWSALFSEYPGFATDSTVALLIGLLFFLIP
AKTLTKTTPTGEIVAFDYSPLITWKEFQSFMPWDIAILVGGGFALADGCE
ESGLSKWIGNKLSPLGSLPAWLIILISSLMVTSLTEVASNPATITLFLPI
LSPLAEAIHVNPLYILIPSTLCTSFAFLLPVANPPNAIVFSYGHLKVIDM
VKAGLGVNIVGVAVVMLGICTWIVPMFDLYTYPSWAPAMSNETMP

Yet another example is the human hypothetical protein XP_{—}091606, which has the following sequence:

(SEQ ID NO:6)
MAGLKAEDAQERLGQREMLGPRLPLEGWPEALESKDASHRRRSPSRAMAS
ALSYVSKFKSFVILFVTPLLLLPLVILMPAKAALRTPGVLCPLTGTDPGS
PELERTDFAGSTALPGRAHSGAPGGTYRKKIITHVDPALSLPLSGPSLGL
SVSIYDPHSFQPGEPHLIHNPAPGCRQFVRCAYVIILMAIYWCTEVIPLA
VTSLMPVLLFPLFQILDSRQVCVQYMKDTNMLFLGGLIVAVAVERWNLHK
RIALRTLLWVGAKPARLMLGFMGVTALLSMWISNTATTAMMVPIVEAILQ
QMEATSAATEAGLELVDKGKAKELPGSQVIFEGPTLGQQEDQERKRLCKA
MTLCICYAASIGGTATLTGTGPNVVLLGQMNELFPDSKDLVNFASWFAFA
FPNMLVMLLFAWLWLQFVYMRFNFKKSWGCGLESKKNEKAALKVLQEETR
KLGPLSFAEINVLICFFLLVILWFSRDPGFMPGWLTVAWVEGETKYVSDA
TVAIFVATLLFIVPSQKPKFNFRSQTEEERKTPFYPPPLLDWKVTQEKVP
WGIVLLLGGGFALAKGSEASGLSVWMGKQMEPLHAVPPAAITLILSLLVA
VFTECTSNVATTTLFLPIFASMSRSIGLNPLYIMLPCTLSASFAFMLPVA
TPPNAIVFTYGHLKVADMVKTGVIMNIIGVFCVFLAVNTWGRAIFDLDHF
PDWANVTHIET

Another example is the human carrier family 13 (sodium/sulfate symporters) member 4 (GenBank NP_{—}036582) which has the following sequence:

(SEQ ID NO:7)
MGLLQGLLRVRKLLLVVCVPLLLLPLPVLHPSSEASCAYVLIVTAVYWVS
EAVPLGAAALVPAFLYPFFGVLRSNEVAAEYFKNTTLLLVGVICVAAAVE
KWNLHKRIALRMVLMAGAKPGMLLLCFMCCTTLLSMWLSNTSTTAMVMPI
VEAVLQELVSAEDEQLVAGNSNTEEAEPISLDVKNSQPSLELIFVNEDRS
NADLTTLMHNENLNGVPSITNPIKTANQHQGKKQHPSQEKPQVLTPSPRK
QKLNRKYRSHHDQMICKCLSLSISYSATIGGLTTIIGTSTSLIFLEHFNN
QYPAAEVVNFGTWFLFSFPISLIMLVVSWFWMHWLFLGCNFKETCSLSKK
KKTKREQLSEKRIQEEYEKLGDISYPEMVTGFFFILMTVLWFTREPGFVP
GWDSFFEKKGYRTDATVSVFLGFLLFLIPAKKPCFGKKNDGENQEHSLGT
EPIITWKDFQKTMPWEIVILVGGGYALASGSKSSGLSTWIGNQMLSLSSL
PPWAVTLLACILVSIVTEFVSNPATITIFLPILCSLSETMHINPLYTLIP
VTMCISFAVMLPVGNPPNAIVFSYGHCQIKDMVKAGLGVNVIGLVIVMVA
INTWGVSLFHLDTYPAWARVSNITDQA

Similar proteins can also be found in other species, e.g., in rodents and so forth.

INDY Polypeptides

The Indy gene codes for a polypeptide or protein having the sequence shown in FIG. 2 (SEQ ID NO:2, GenBank accession no. AE003519), such sequence having substantial homology with a Drosophila gene (accession no. AF217399), human sodium dicarboxylate cotransporters (hNaDC-1, accession No. U26209) and rat sodium dicarboxylate cotransporters (SDCT2, accession no. AF081825, and SDCT1, accession no. AF058714). The Indy gene product and the family of cellular transporters in mammals appears to define a new class of gene products involved in determining life span and metabolic control.

Accordingly in another embodiment, there is provided a substantially pure polypeptide homologous to SEQ ID NO:2. A “substantially pure polypeptide” is an INDY polypeptide that has been separated from components that naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, INDY polypeptide. A substantially pure INDY polypeptide may be obtained, for example, by extraction from a natural source (e.g., an insect cell); by expression of a recombinant nucleic acid encoding an INDY polypeptide; or by chemically synthesizing the protein. Purity can be measured by any appropriate method, e.g., by column chromatography, polyacrylamide gel electrophoresis, or by HPLC analysis.

INDY Mutagenesis

Amino acids essential for the function of INDY polypeptides can be identified according to procedures known in the art, such as site-directed mutagenesis or alanine-scanning mutagenesis (Cunningham and Wells, Science 244: 1081-1085, 1989; Bass et al., Proc. Natl. Acad. Sci. USA 88: 4498-4502, 1991). In the latter technique, single alanine mutations are introduced at different residues in the molecule, and the resultant mutant molecules are tested for biological activity (e.g., ligand binding and signal transduction) to identify amino acid residues that are critical to the activity of the molecule. Sites of ligand-protein interaction can also be determined by analysis of crystal structure as determined by such techniques as nuclear magnetic resonance, crystallography or photoaffinity labeling. (See, for example, de Vos et al., Science 255: 306-312, 1992; Smith et al., J. Mol. Biol. 224: 899-904, 1992; Wlodaver et al., FEBS Lett. 309: 59-64, 1992). The identities of essential amino acids can also be inferred from analysis of homologies with related proteins.

Multiple amino acid substitutions can be made and tested using known methods of mutagenesis and screening, such as those disclosed by Reidhaar-Olson and Sauer, Science 241: 53-57,1988; or Bowie and Sauer, Proc. Natl. Acad. Sci. USA 86: 2152-2156, 1989. Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30: 10832-10837, 1991; Ladner et al., U.S. Pat. No. 5,223,409; Huse, WIPO Publication WO 92/06204) and region-directed mutagenesis (Derbyshire et al., Gene 46: 145, 1986; Ner et al., DNA 7: 127, 1988).

Mutagenesis methods as disclosed above can be combined with high-throughput screening methods to detect the activity of cloned, mutagenized proteins in host cells. Mutagenized DNA molecules that encode active proteins or portions thereof (e.g., ligand-binding fragments) can be recovered from the host cells and rapidly sequenced using modem equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptides that are substantially homologous to SEQ ID NO:2 or allelic variants thereof and retain the properties of the wild-type polypeptide. As expressed and claimed herein the language, “a polypeptide as defined by SEQ ID NO: 2” includes all allelic variants and species orthologs of the polypeptide. The term “polypeptide” as used herein includes modified sequences such as glycoproteins, and is specifically intended to cover naturally occurring polypeptides or proteins, as well as those that are recombinantly or synthetically synthesized, which occur in at least two different conformations wherein both conformations have the same or substantially the same amino acid sequence but have different three dimensional structures. “Fragments” are a portion of a naturally occurring protein. Fragments can have the same or substantially the same amino acid sequence as the naturally occurring protein.

INDY polypeptides and peptide fragments including mutated, truncated or deleted forms can be prepared for a variety of uses including generation of antibodies, as reagents in diagnostic assays, for the identification of other gene products involved in the regulation of life span and body weight, as reagents for screening for compounds that can be used in the extension of life span or body weight control and as pharmaceutical treatments useful for extension of lifespan or for treatment of body weight disorders.

The disclosure also encompasses proteins that are functionally equivalent to the Indy gene product, as judged by any of a number of criteria, including but not limited to the resulting biological effect of Indy, for example, a change in phenotype such as an alteration of life span when the Indy equivalent is present in an appropriate cell type. Such functionally equivalent INDY proteins include additions or substitutions of amino acid residues within the amino acid sequence encoded by the Indy nucleotide sequences described, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Anti-INDY Antibodies

In another embodiment, the invention comprises antibodies that specifically recognize one or more epitopes of INDY or conserved variants of INDY or fragments of INDY. Such antibodies may be polyclonal antibodies, monospecific (e.g., monoclonal) antibodies, humanized or chimeric antibodies, anti-idiotypic antibodies, single chain antibodies, Fab fragments, fragments produced from an Fab expression library, and epitope-binding fragments of the above.

The term “antibody” as used herein refers to an immunoglobulin molecule or immunologically active portion thereof, i.e., an antigen-binding portion. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

Antibodies that bind to the Drosophila INDY polypeptide can be prepared from the intact polypeptide or fragments containing peptides of interest as the immunizing agent. A preferred INDY polypeptide fragment is 15-30 contiguous amino acids of SEQ ID NO:2. In one embodiment, the peptide is located in a non-transmembrane domain of the INDY polypeptide, e.g., in an extracellular or intracellular loop. Thus, antibodies that bind to an epitope of Drosophila INDY, e.g., located within amino acid residues 1-25, 41-65, 68-96, 86-98, 114-135, 153-210, 229-256, 272-316, 334-355, 375-398, 418-444, 461-485, 502-531, and 541-572 of SEQ ID NO:2, are within the scope of the invention. An exemplary antibody or antibody fragment binds to an epitope that is accessible from the extracellular milieu and that alters the functionality of the transporter. Such proteins can be identified, e.g., using methods for preparing immunoglobulins and methods for assaying transporters described herein. Similar immunoglobulin proteins can be identified that alter the functionality of a mammalian carboxylate transporter, e.g., a mammalian carboxylate transporter described herein.

The preparation of polyclonal antibodies is well known in the molecular biology art; see for example, Production of Polyclonal Antisera in Immunochemical Processes (Manson, ed.), pages 1-5 (Humana Press 1992) and Coligan et al., Production of Polyclonal Antisera in Rabbits, Rats, Mice and Hamsters in Current Protocols in Immunology, section 2.4.1 (1992). The preparation of monoclonal antibodies is also well known in the art; see for example, Harlow et al., Antibodies: A Laboratory Manual, page 726 (Cold Spring Harbor Pub. 1988). Briefly, monoclonal antibodies can be obtained by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods.

Phage display and combinatorial methods can be used to isolate recombinant antibodies that bind to INDY or fragments thereof (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982).

Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

A therapeutically useful anti-INDY antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, 1986 and Singer et al., J. Immunol. 150: 2844, 1993. The antibodies can also be derived from human antibody fragments isolated from a combinatorial immunoglobulin library; see, for example, Barbas et al., Methods: A Companion to Methods in Enzymology 2, 119, 1991.

In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies against Indy gene products, as described, for example, in U.S. Pat. No. 4,946,778. Single chain antibodies are formed by linking the heavy and light chain fragments of the Fv region via an amino acid bridge, resulting in a single chain polypeptide.

Antibody fragments that recognize specific epitopes, e.g., extracellular epitopes, can be generated by techniques well known in the art. Such fragments include Fab fragments produced by proteolytic digestion, and Fab fragments generated by reducing disulfide bridges.

When used for immunotherapy, the monoclonal antibodies, fragments thereof, or both, that bind to INDY may be unlabelled or labeled with a therapeutic agent. These agents can be coupled directly or indirectly to the monoclonal antibody by techniques well known in the art, and include such agents as drugs, radioisotopes, lectins and toxins.

The monoclonal antibodies can be used alone or in combination with therapeutic agents such as those described above. Preferred combinations include monoclonal antibodies that bind INDY and immunomodulators and other biological response modifiers. The dosage ranges for the administration of monoclonal antibodies are large enough to produce the desired effect, either a change in life span or change in body weight. The dosage will vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, subcutaneously, intracavity, or transdermally, alone or with effector cells.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

Uses of Indy

The novelty in the discovery of the Drosophila Indy gene lies in the fact that Indy mutants may be genetically calorically restricted, allowing them to eat normally, maintain high levels of physical activity and reproductive status, while benefiting from increased life span. The discovery of Indy mutant animals has identified a target to which appropriate therapeutic agents could be designed to provide a chemical intervention. Such drugs could potentially block the uptake of key metabolites by INDY protein, and, at appropriate doses, provide the benefit of increased longevity through a form of caloric restriction. Such INDY-based agents would also have potential benefit in the control of ideal body weight. Because the INDY protein is an accessible target (for instance, it may have a primary role in the gut in absorbing, utilizing and/or storing metabolites), such INDY-drugs could be designed to have low absorption/toxicity affects and potentially exert their largest effects in a non-invasive, controlled ambush of a fraction of a person's intake of nutrients. There may also be undiscovered naturally occurring substances that block INDY function.

Without being bound by theory, it appears that the Indy gene encodes a cellular transporter that transports key intermediates of the Krebs cycle (i.e., citric acid cycle). The transported intermediates include organic carboxylates, more particularly substituted and unsubstituted dicarboxylates having from two to about ten, preferably four to about six carbon atoms such as succinate, alpha-ketoglutarate and fumarate; and substituted and unsubstituted tricarboxylates having from three to about ten, preferably four to about carbon atoms, for example citrate. Suitable substitutions include but are not limited to hydroxyl groups, carbonyl groups, sulfhydryl groups, and the like. Unsaturation may also be present. A preferred substrate for INDY is succinate. It is to be understood that although reference is made to the ionized form of the acids, the protonated acid or another conjugated form may actually be transported. INDY is thus described as a cellular transporter of carboxylates, particularly di- and tricarboxylates.

Experimental evidence indicates that unlike other transporters of Krebs cycle intermediates (the sodium dicarboxylate cotransporters, for example), INDY does not appear to require monovalent cations to transport its substrates. INDY is thus referred to herein as a “transporter”. However, this term does not exclude any form of the Indy gene product that in fact co-transports other moieties along with the carboxylates, for example divalent cations.

The Aging Process

All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

In some cases, characteristics of aging can be quite obvious. For example, characteristics of older humans include skin wrinkling, graying of the hair, baldness, and cataracts, as well as hypermelanosis, osteoporosis, cerebral cortical atrophy, lymphoid depletion, thymic atrophy, increased incidence of diabetes type II, atherosclerosis, cancer, and heart disease. Nehlin et al. (2000), Annals NY Acad Sci 980:176-79. Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoictic precursor cell ablation), liver pathology, atrophy of intestinal villi, skin ulceration, amyloid deposits, and joint diseases. Tyner et al. (2002), Nature 415:45-53.

Careful observation reveals characteristics of aging in other eukaryotes, including invertebrates. For example, characteristics of aging in the model organism C. elegans include slow movement, flaccidity, yolk accumulation, intestinal autofluorescence (lipofuscin), loss of ability to eat food or dispel waste, necrotic cavities in tissues, and germ cell appearance.

Those skilled in the art will recognize that the aging process is also manifested at the cellular level, as well as in mitochondria. Cellular aging is manifested in loss of doubling capacity, increased levels of apoptosis, changes in differentiated phenotype, and changes in metabolism, e.g., decreased levels of protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to oxidative stress, rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid metabolism, ribosomal translation rates, etc.). As used herein, “biological age” is a measure of the age of a cell or organism based upon the molecular characteristics of the cell or organism. Biological age is distinct from “temporal age”, which refers to the age of a cell or organism as measured by days, months, and years.

Those skilled in the art will recognize that the process of aging is distinct from diseases that reduce life span and are correlated with aging, such as cancer, heart disease or diabetes. Unlike the diseases that reduce life span, aging is a genetic program (subject to some environmental input) that determines life span. Providing a cure for a disease like cancer will allow an organism to live out their normal life span (barring other diseases, of course). In contrast, by identifying the cellular regulators of life span, it will be possible to design compounds that modulate the activity of said cellular regulators to thereby truly extend the life span of organism.

Screens

Described below are exemplary methods for identifying compounds that can reduce the rate of aging of an organism and thereby slow or ameliorate the pathologies associated with increased temporal age. The Indy mutants that increase the life span of flies and reduce their rate of aging are loss of function mutations. Accordingly, the expression or activity of an Indy gene product in an organism can be a determinant of the rate of aging and life span of the organism. Reduction in the level and/or activity of such gene products would reduce the rate of aging and ameliorate (at least temporarily) the symptoms of aging. A variety of techniques may be utilized to inhibit the expression, synthesis, or activity of such target genes and/or proteins. Such molecules may include, but are not limited to small organic molecules, peptides, antibodies, antisense, ribozyme molecules, triple helix molecules, and the like.

The following assays provide methods (also referred to herein as a “screening assay”) for identifying modulators, i.e., candidate or test compounds or agents (e.g., peptides, peptidomimetics, small molecules or other drugs) which bind to the INDY polypeptide, have a stimulatory or inhibitory effect on, for example, Indy expression or INDY activity, or have a stimulatory or inhibitory effect on, for example, the expression or activity of an INDY substrate. Such compounds can be agonists or antagonists of INDY function.

Some exemplary screening assays for assessing transporter function or state include one or more of the following features:

use of ¹⁴C-labeled substrates of the transporters, e.g., where the substrates are ¹⁴C-labeled succinate, fumarate, alpha-ketoglutarate, malate, citrate, oxaloacetate or glutaric acid (¹⁴C=carbon 14);

use of ³H-labeled substrates of the transporters, e.g., where the substrates are ³H-labeled succinate, fumarate, alpha-ketoglutarate, malate, citrate, oxaloacetate or glutaric acid (³H=tritium);

use of a transgenic cell, e.g., with a transgene encoding a heterologous transporter;

use of a mammalian cell that expresses the transporter;

use of an oocyte, e.g., a Xenopus oocyte;

use of an artificial lipid bilayer;

detection of binding or transport of a fluorescent-labeled substrate, e,g, succinate, fumarate, alpha-ketoglutarate, malate, citrate, oxaloacetate or glutaric acid;

detection of binding of a fluorescent-labeled compound where the compound is, for example, a peptide, protein, antibody or small organic molecule;

detection of binding of an ¹²⁵I-labeled compound peptides, proteins, antibodies or other small organic molecules

detection of ion flux, e.g., using electrophysiology, a pH detection (e.g., using a pH sensitive dye or direct pH detection), voltage detection (e.g., using a voltage sensitive dye or direct voltage detection,), Ca2+ flux (e.g., using a Ca2+ sensitive dye), the detection can be, e.g., in mammalian cells, oocytes expressing the transporter, a transgenic cell;

use of fluorescence resonance energy transfer (FRET) of labeled heterologous transporters; and

use of fluorescence proximity assays of labeled heterologous transporters.

One embodiment herein accordingly comprises methods for the identification of small molecule drug candidates from large libraries of compounds that appear to have therapeutic activity to affect metabolic maintenance and/or to reverse or prevent cell death and thus exhibits potential therapeutic utility enhancing longevity. Small organic molecules and peptides having effective inhibitory activity may be designed de novo, identified through assays or screens, or obtained by a combination of the two techniques. Non-protein drug design may be carried out using computer graphic modeling to design non-peptide, organic molecules able to bind to the cellular transporters. The use of nuclear magnetic resonance (NMR) data for modeling is also known in the art, as described by Lam et al., Science 263: 380, 1994, using information from x-ray crystal structure studies of the transporter.

Small molecules may also be developed by generating a library of molecules, selecting for those molecules which act as ligands for a specified target, (using protein functional assays, for example), and identifying the selected ligands. See, e.g., Kohl et al., Science 260: 1934, 1993. Techniques for constructing and screening combinatorial libraries of small molecules or oligomeric biomolecules to identify those that specifically bind to a given receptor protein are known. Suitable oligomers include peptides, oligonucleotides, carbohydrates, nonoligonucleotides (e.g., phosphorothioate oligonucleotides; see Chem. and Engineering News, page 20, Feb. 7, 1994) and nonpeptide polymers (see, e.g., “peptoids” of Simon et al., Proc. Natl. Acad. Sci. USA 89 9367, 1992). See also U.S. Pat. No. 5,270,170 to Schatz; Scott and Smith, Science 249: 386-390, 1990; Devlin et al., Science 249: 404-406, 1990; Edgington, BI0/Technology, 11: 285, 1993. Libraries may be synthesized in solution on solid supports, or expressed on the surface of bacteriophage viruses (phage display libraries).

Known screening methods may be used by those skilled in the art to screen combinatorial libraries to identify active molecules. For example, an increase (or decrease) in active uptake of a nutrient due to contact with a transporter agonist or antagonist can be monitored.

In one embodiment, assays for screening candidate or test compounds that are substrates of an INDY protein or polypeptide or biologically active portion thereof are provided. In another embodiment, assays for screening candidate or test compounds which bind to or modulate the activity of an INDY protein or polypeptide or biologically active portion thereof, e.g., modulate the ability of INDY to interact with a ligand, are provided. In still another embodiment, assays for screening candidate or test compounds for the ability to bind to or modulate the activity of an INDY protein or polypeptide and to also alter the rate of aging of a cell or an organism are provided.

Examples of methods for the synthesis of molecular libraries can be found in the art, for example in: DeWitt et al., Proc. Natl. Acad. Sci. U.S.A. 90: 6909, 1993; Erb. et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and in Gallop et al., J. Med. Chem. 37:1233, 1994.

Libraries of compounds may be presented in solution (e.g., Houghten, Biotechniques 13: 412-421, 1992), or on beads (Lam, Nature 354: 82-84, 1991), chips (Fodor, Nature 364: 555-556, 1993), bacteria (Ladner U.S. Pat. No. 5,223,409), spores (Ladner U.S. Pat. No. '409), plasmids (Cull et al., Proc Natl Acad Sci USA 89: 1865-1869, 1992) or on phage (Scott and Smith, Science 249: 386-390, 1990); (Devlin, Science 249: 404-406, 1990); (Cwirla et al., Proc. Natl. Acad. Sci U.S.A. 87: 6378-6382, 1990); (Felici, J. Mol. Biol. 222: 301-310, 1991); (Ladner supra.).

The compounds tested as modulators of a transporter protein can be any small chemical compound, or a biological entity, such as a protein, e.g., an antibody, a sugar, a nucleic acid, e.g., an antisense oligonucleotide or a ribozyme, or a lipid. Alternatively, modulators can be genetically altered versions of a transporter protein. Typically, test compounds will be small chemical molecules and peptides, or antibodies, antisense molecules, or ribozymes. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. Moreover, a combinatorial library can be designed to sample a family of compounds based on a parental compound, e.g., based on the chemical structure of 4,4′-Diisothyocyanostilbene-2,2′-disulfonic acid (DIDS).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, e.g., where each assay includes a cell or tissue expressing a carboxylate transporter. In a high throughput assays, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100- about 1500 different compounds. It is possible to assay many plates per day; assay screens for up to about 6,000, 20,000, 50,000, or 100,000 or more different compounds are possible using the integrated systems of the invention.

Candidate INDY interacting molecules encompass many chemical classes. They can be organic molecules, preferably small organic compounds having molecular weights of 50 to 2,500 Daltons. The candidate molecules comprise functional groups necessary for structural interaction with proteins, particularly hydrogen bonding, for example, carbonyl, hydroxyl, and carboxyl groups. The candidate molecules can comprise cyclic carbon or heterocyclic structures and aromatic or polyaromatic structures substituted with the above groups. In one embodiment, the candidate molecules are structurally and/or chemically related to 4,4′-diisothyocyanostilbene-2,2′-disulfonic acid (DIDS), e.g., that superimpose on a model of the structure of 4,4′-diisothyocyanostilbene-2,2′-disulfonic acid (DIDS) with a root-mean-squared-standard deviation (RMSD) of less than about 2, 1.5, or 1 Angstroms.

Other techniques are known in the art for screening synthesized molecules to select those with the desired activity, and for labeling the members of the library so that selected active molecules may be identified, as in U.S. Pat. No. 5,283,173 to Fields et al., (use of genetically altered Saccharomyces cerevisiae to screen peptides for interactions). As used herein, “combinatorial library” refers to collections of diverse oligomeric biomolecules of differing sequence, which can be screened simultaneously for activity as a ligand for a particular target. Combinatorial libraries may also be referred to as “shape libraries”, i.e., a population of randomized fragments that are potential ligands. The shape of a molecule refers to those features of a molecule that govern its interactions with other molecules, including Van der Waals, hydrophobic, electrostatic and dynamic.

Nucleic acid molecules may also act as ligands for receptor proteins. See, e.g., Edgington, BIO/Technology 11: 285, 1993. U.S. Pat. No. 5,270,163 to Gold and Tuerk describes a method for identifying nucleic acid ligands for a given target molecule by selecting from a library of RNA molecules with randomized sequences those molecules that bind specifically to the target molecule. A method for the in vitro selection of RNA molecules immunologically cross-reactive with a specific peptide is disclosed in Tsai, Kenan and Keene, Proc. Natl. Acad. Sci. USA 89:8864, 1992; and Tsai and Keene, J. Immunology 150:1137, 1993. In the method, an antiserum raised against a peptide is used to select RNA molecules from a library of RNA molecules; selected RNA molecules and the peptide compete for antibody binding, indicating that the RNA epitope functions as a specific inhibitor of the antibody-antigen interaction.

Antibodies that are both specific for target gene protein and interfere with its activity may be used to inhibit target gene function. Such antibodies may be generated using standard techniques, against the proteins themselves or against peptides corresponding to portions of the proteins. Such antibodies include but are not limited to polyclonal, monoclonal, Fab fragments, single chain antibodies, chimeric antibodies, and the like. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Sambrook et al., Eds., Molecular Cloning: A Laboratory Manual, 2nd ed., Cold Spring Harbor Laboratory Press, 1989, or Ausubel, F. M. et al., eds. Current Protocols in Molecular Biology, 1994).

Alternatively, single chain neutralizing antibodies that bind to intracellular target gene epilopes may also be administered. Such single chain antibodies may be administered, for example, by expressing nucleotide sequences encoding single-chain antibodies within the target cell population by utilizing, for example, techniques such as those described in Marasco et al., Proc. Natl. Acad. Sci. USA 90: 7889-7893, 1993.

Also encompassed are assays for cellular proteins that interact with INDY. Any method suitable for detecting protein-protein interactions may be used. The traditional methods that may be used include, for example, co-immunoprecipitation, crosslinking, and co-purification through gradients or chromatographic columns. For these assays, Indy can be a full-length protein or an active fragment. Additional methods include those methods that allow for the simultaneous identification of genes that encode proteins that interact with INDY. These methods include, for example, probing expression libraries using a labeled INDY protein, INDY fragment, or INDY fusion protein.

One method to detect protein-protein interaction in vivo is the two-hybrid system, see, for example, Chien et al., Proc. Natl. Acad. Sci, USA 88: 9578-9582, 1991. In brief, the two-hybrid system utilizes plasmids constructed to encode two hybrid proteins: one plasmid comprises the nucleotides encoding the DNA binding domain of a transcriptional activator protein fused to the Indy nucleotide sequence encoding the INDY polypeptide, and the other plasmid comprises the nucleotides encoding the transcriptional activator protein's activation domain fused to a cDNA encoding an unknown protein that has been recombined into the plasmid from a cDNA library. The DNA binding domain fusion plasmid and the cDNA fusion protein library plasmids are transformed into a strain of yeast that contains a reporter gene, for example lacZ, whose regulatory region contains the activator's binding site. Either hybrid protein alone cannot activate translation of the reporter gene because it is lacking either the DNA binding domain or the activator domain. Interaction of the two hybrid proteins, however, reconstitutes a functional activator protein and results in activation of the reporter gene that is detected by an assay for the reporter gene product. The colonies that reconstitute activator activity are purified and the library plasmids responsible for reporter gene activity are isolated and sequenced. The DNA sequence is then used to identify the protein encoded by the library plasmid.

Macromolecules that interact with INDY are referred to as Indy binding partners. Indy binding partners are likely to be involved in the regulation of INDY function. Therefore, it is possible to identify compounds that interfere with the interaction between INDY and its binding partners. The basic principle of assay systems used to identify compounds that interfere with the interaction of Indy and a binding partner is to prepare a reaction mixture containing INDY or an INDY fragment and the binding partner under conditions that allow complex formation. The reaction mixture is prepared in the presence or absence of the test compound to test for inhibitory activity. The test compound may be added prior to or subsequent to INDY/binding partner complex formation. The formation of a complex in a control but not with the test compound confirms that the test compound interferes with complex formation. The assay can be conducted either in the solid phase or in the liquid phase.

In another embodiment, an assay is a cell-based assay comprising contacting a cell expressing INDY with a test compound and determining the ability of the test compound to modulate (e.g. stimulate or inhibit) the activity of INDY. A preferred activity is the transporter function of INDY. Determining the ability of the test compound to modulate the activity of INDY can be accomplished, for example, by determining the ability of INDY to bind to or interact with the test molecule, or by determining the ability of the test molecule to stimulate or inhibit the transporter function of INDY. Cell-based systems can be used to identify compounds that inhibit INDY. Such cells can be recombinant or non-recombinant, such as cell lines that express the Indy gene. Preferred systems are Xenopus oocytes containing the Indy mRNA and yeast cells that express Indy. In utilizing such systems, cells are exposed to compounds suspected of ameliorating body weight disorders or increasing lifespan. After exposure, the cells are assayed, for example, for expression of the Indy gene or the INDY protein. Alternatively, the cells are assayed for phenotypes such as those resembling body weight disorders or lifespan extension. The cells may also be assayed for the inhibition of the transporter function of INDY.

A preferred cell for use in a cell-based assay comprises a Xenopus oocyte containing the Indy mRNA. Such Xenopus oocytes will express the INDY polypeptide and can be used to study the transporter function of INDY. A Xenopus oocyte expressing the INDY polypeptide is useful for screening test compounds for alteration in INDY function. Compounds that either increase or decrease the transport of Krebs cycle intermediates by INDY can be identified in this system.

Another preferred cell for a cell-based assay comprises a yeast cell transformed with a vector comprising the Indy gene. One use for a yeast cell expressing Indy is to mutagenize the yeast and screen for yeast that will survive only when the INDY polypeptide is functioning normally. Synthetic lethal screens are described in Holtzman et al. (1993), J. Cell Bio. 122: 635-644. The yeast that require Indy function for survival can then be used to screen test compounds for those that inhibit Indy activity. Test compounds that results in a decrease in yeast survival are likely inhibitors of INDY in this system.

In yet another embodiment, an assay is a cell-free assay in which an INDY protein or biologically active portion thereof is contacted with a test compound and the ability of the test compound to bind to the INDY protein or biologically active portion thereof is determined. Binding of the test compound to the INDY protein can be determined either directly or indirectly as described above. In a preferred embodiment, the assay includes contacting the INDY protein or biologically active portion thereof with a known compound which binds INDY to form an assay mixture, contacting the assay mixture with a test compound, and determining the ability of the test compound to interact with an INDY protein, wherein determining the ability of the test compound to interact with an INDY protein comprises determining the ability of the test compound to preferentially bind to INDY or a biologically active portion thereof as compared to the known compound.

The cell-free assays are amenable to use of both soluble and/or membrane-bound forms of proteins. In the case of cell-free assays in which a membrane-bound form of the protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPS O), or N-dodecyl,N,N-dimethyl-3-ammonio-1-propane sulfonate.

In more than one embodiment of the above assay methods, it may be desirable to immobilize either INDY or its target molecule to facilitate separation of complexed from uncomplexed forms of one or both of the proteins, as well as to accommodate automation of the assay. Binding of a test compound to an INDY protein, or interaction of an INDY protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/INDY fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or INDY protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of INDY binding or activity determined using standard techniques.

Other techniques for immobilizing proteins on matrices can also be used in the screening assays of the invention. For example, either an INDY protein or an INDY target molecule can be immobilized utilizing conjugation of biotin and streptavidin. Biotinylated INDY protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with INDY protein or target molecules but which do not interfere with binding of the INDY protein to its target molecule can be derivatized to the wells of the plate, and unbound target INDY protein trapped in the wells by antibody conjugation. Methods for detecting such complexes, in addition to those described above for the GST-immobilized complexes, include immunodetection of complexes using antibodies reactive with the INDY protein or target molecule, as well as enzyme-linked assays which rely on detecting an enzymatic activity associated with the INDY protein or target molecule.

In addition to cell-based and in vitro assay systems, non-human organisms, e.g., transgenic non-human organisms, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g., mice, rats, rabbits, guinea pigs, pigs, micro-pigs, and goats), and non-human primates (e.g., baboons, monkeys, chimpanzees) may be used in the methods of the invention.

Accordingly, in another embodiment, the invention features a method of identifying a compound that alters the rate of aging of a cell or an organism, comprising: contacting a carboxylate transporter polypeptide with a test compound; evaluating an interaction between the test compound and the carboxylate transporter polypeptide; and further evaluating the effect of the test compound on the rate of aging of a cell or organism.

The interaction between a test compound and the carboxylate transporter polypeptide can be performed by any of the methods described herein, e.g., using cell-based assays or cell-free in vitro assays. Weather the interaction between the test compound and the carboxylate transporter polypeptide is evaluated prior to the evaluation of the effect of the text compound on the rate of aging of a cell or organism is not critical to the method. However, it is preferable to evaluate the interaction between the test compound and carboxylate transporter polypeptide first, so that test compounds that do not interact with the carboxylate transporter polypeptide do not have to be tested for their effect upon the rate of aging. It can also be preferable to use an assay for evaluating the interaction between the test compound and the carboxylate transporter polypeptide that can be adapted for high throughput screening, thus making it possible to screen one or more libraries of test compounds. Possible test compounds include, e.g., small organic molecules, peptides, antibodies, and nucleic acid molecules, as described above.

The carboxylate transporter polypeptide can have greater than or equal to 25% overall identity or greater than or equal to 30% overall similarity to SEQ ID NO:2. Similarly, it can be a cation independent carboxylate transporter polypeptide. Preferably, the carboxylate transporter polypeptide is a human protein (e.g., as described herein), although it may also be desirable to analyze carboxylate transporter polypeptides isolated from other organisms such as yeast, worms, flies, fish, reptiles, birds, mammals (especially rodents), and primates using the methods of the invention.

The rate of aging of an organism can be determined using methods known in the art. For example, the rate of aging of an organism can be determined by directly measuring the life span of the organism. Preferably, a statistical measure, e.g., an average or median value, of the life span of a group of animals, e.g., a group of genetically matched animals, will be determined and the resulting statistical value compared to an equivalent statistical value, e.g, an average of median value, of the life span of a control group of animals, e.g., a group of animals that did not receive the test compound but are genetically matched to the group of animals that did receive the test compound. Such methods are suitable for organisms that have a short life span, such as worms or flies. See, for example, Rogina et al. (2000), Science 290:2137-40. Direct measurement of life span can also be preformed with other organisms such as rodents, as discussed, for example, in Weindruch et al. (1986), Journal of Nutrition 116(4):641-54. Those skilled in the art will recognize that there are many ways of measuring the statistical difference between two sets of data, any of which may be suitable for the methods of the invention.

To reduce the time that it takes to measure a change in the rate of aging using data on the life span of the organisms treated with the test compound, various modifications or treatments of the organisms can be implemented. For example, animals fed on a calorically rich diet tend to live shorter lives, thus reducing the time that needs to elapse to determine when the average life span of the test group of animals has exceeded the average life span of the control group of animals. Alternatively, the test compound can be administered to test animals that have already lived for 50%, 60%, 70%, 80%, 90%, or more of their expected life span. Thus, the test compound can be administered to an adult organism, or even an old adult organism. Other possibilities include the use of genetically modified organisms. For example, the organisms could harbor mutations (e.g., a Hyperkinetic¹ or Shaker⁵ mutation in Drosophila, or a mutation in a silent information regulator gene (e.g., Sir2), or a catalase or superoxide dismutase gene) or transgenes (e.g., encoding a transporter protein (e.g., a carboxylate transport protein such as INDY) or a protein involved in insulin signaling and metabolic regulation (e.g., IGF-1)) that reduce their average life span. See Rogina et al. (1997), Proc. Natl. Acad. Sci., USA 94:6303-6; Rogina and Helfand (2000), Biogerontology 1:163-9; and Guarente and Kenyon (2000), Nature 408:255-62. Those skilled in the art will understand that it may also be desirable to practice the methods of the invention using organisms that are long-lived, such as calorically restricted animals, or animals carrying mutations or transgenes that increase their life span.

A proxy for rate of aging of a cell or an organism can be determined using biomarkers that are indicative of the biological age of the organism (i.e., age-related parameters). Using biomarkers for determining biological age can greatly facilitate screens for compounds that alter the rate of aging, as they bypass the requirement of waiting for the animal to die in order to determine the rate of aging. The expression of genes whose regulation is biological age-dependent are preferred biomarkers for use in the methods of the invention. Numerous genes are known to be expressed in a biological age-dependent manner. In Drosophila, for example, such genes include wingless and engrailed. See Rogina and Helfand (1997), Mechanisms of Development 63:89-97. In mice, the expression of the ras oncogene is elevated in older animals. See Hass et al. (1993), Mutat. Res. 295(4-6):281-9. Similarly, in rodents and worms, genes that differentially expressed in young and old organisms have been identified by transcriptional profiling using microarrays. See, e.g., Lee et al. (1999), Science 285:1390-93; WO 01/12851; and Hill et al. (2000), Science 290:809-812. For example, Hill et al. (2000) Science 90:809 discloses genes whose transcripts are up-regulated in nematodes that are at 2 weeks in development. Examples of such genes include the genes described in cluster (4,1):69 of Hill, supra. Any gene whose regulation is biological age-dependent is suitable for the methods of the invention. Preferably, more than one gene is analyzed so as to improve the accuracy of the determination. Analysis of gene expression can be performed by any technique known in the art, including Northern, in-situ hybridization, quantitative PCR, and transcriptional profiling using microarrays. Methods of determining biological age based on gene expression patterns are described in WO 01/12851.

Metabolic parameters can also be used to evaluate the rate of aging of a cell or organism. For example, the rate of protein synthesis and degradation decreases in biologically aged cells, and the levels proteins having advanced glycosylation end product modifications increases. See, Lambert and Merry (2000), Exp. Gerontol 35(5):583-94; and WO 01/79842. In addition, animals that harbor mutations conferring longer life span (and thus a reduced rate of aging) can show defects in ubiquinone biosynthesis, mitochondrial biogenesis, glucose metabolism, nucleic acid metabolism, ribosomal translation rates, and cholesterol biosynthesis. See, for example, WO 98/17823 and WO 99/10482. Thus, by measuring any of these parameters or some combination thereof, it is possible to indirectly evaluate the rate of aging of a cell or an organism. Methods of analyzing protein synthesis, degradation, and modification with advanced glycosylation end products are known in the art, as described in Lambert and Merry (2000), Exp. Gerontol 35(5):583-94 and WO 01/79842. Similarly, methods of analyzing ubiquinone biosynthesis, mitochondrial biogenesis, and glucose metabolism are known in the art (see, e.g., Marbois et al. J. Biol. Chem. 271:2995; Proft et al. EMBO J. 14:6116; and WO 98/17823), as are methods of analyzing nucleic acid metabolism, ribosomal translation rates, and cholesterol biosynthesis (see, e.g., WO 99/10482).

Cellular proliferation is another parameter that can be used to evaluate the biological age of a cell or organism. Cells from biologically aged organisms demonstrate reduced proliferative capacity as compared to the cells of a corresponding younger organism. See Li et al. (1997), Invest. Ophthalmol. 38(1):100-7; and Wolf and Pendergrass (1999), J Gerontol. A Biol. Sci. Med. Sci. 54(11):B502-17. It will be understood by one skilled in the art that there are many methods for evaluating the proliferative capacity of cells that are suitable for use in the methods of the invention. For example, cells can be labeled in vitro (or in vivo) with BrdU to determine the percent of dividing cells or evaluated using a colony forming assay, as described in Li et al. (1997), supra. Cell suitable for the analysis of proliferative capacity include cells grown in tissue culture, cells isolated from an animal that has been treated with a test compound, cells that are part of a live animal, or cells that are part of a tissue section obtained from an animal. With respect to cells present in an animal or tissue section thereof, preferable cells include lens epithelial cells, osteoblasts, osteoclasts, and lymphoid cells.

Basically, any biomarker that is altered in a biological age-dependent manner has the potential to be used to evaluate the effect of a test compound upon the rate of aging of a cell or an organism. Thus, additional biomarkers include visual appearance, resistance to oxidative stress, cellular transformation (the ability to adopt a transformed (i.e., cancerous or malignant) phenotype), or DNA methylation (e.g., of a ras oncogene). See, for example, Finkel and Holbrook (2000), Nature 408:239-47; Kari et al. (1999), J Nutr. Health Aging 3(2):92-101; and Hass et al. (1993), Mutat. Res. 295(4-6):281-9.

A cell used in the methods of the invention can be from a stable cell line or a primary culture obtained from an organism, e.g., a organism treated with the test compound.

A transgenic cell or animal used in the methods of the invention can include a transgene that encodes, e.g., a copy of a carboxylate transport protein, e.g., the carboxylate transporter polypeptide that was evaluated for an interaction with the test compound. The transgene can encode a protein that is normally exogenous to the transgenic cell or animal, including a human protein, e.g., a human carboxylate transporter polypeptide. The transgene can be linked to a heterologous or a native promoter.

Transgenic Organisms

This disclosure further relates to a method of producing transgenic animals, e.g., mice or flies. In one embodiment, the transgenic animal is engineered to express, overexpress or ectopically express a carboxylate transporter, e.g., Drosophila Indy, which method comprises the introduction of several copies of a segment comprising at least the polynucleotide sequence encoding SEQ ID NO:2 with a suitable promoter into the cells of an embryo at an early stage. Techniques known in the art may be used to introduce the Indy transgene into animals to produce the founder line of animals. Such techniques include, but are not limited to: pronuclear microinjection (U.S. Pat. No. 4,873,191); retrovirus mediated gene transfer into germ lines (Van der Putten et al., Proc. Natl. Acad. Sci. USA 82: 6148-6152, 1985; gene targeting in embryonic stem cells (Thompson et al., Cell 56: 313-321, 1989; electroporation of embryos (Lo, Mol. Cell Biol. 3: 1803-1814, 1983; and sperm-mediated gene transfer (Lavitrano, et al., Cell 57: 717-723, 1989; etc. For a review of such techniques, see Gordon, Intl. Rev. Cytol. 115: 171-229, 1989.

Gene targeting by homologous recombination in embryonic stem cells to produce a transgenic animal with a mutation in the Indy gene (“knock-out” mutation) can also be performed. In such so-called “knock-out” animals, there is inactivation of the Indy gene or altered gene expression, such that the animals can be useful to study the function of the Indy gene, thus providing animals models of human disease, which are otherwise not readily available through spontaneous, chemical or irradiation mutagenesis.

A particularly useful transgenic animal in one in which the Indy homolog has been disrupted or knocked out. Analysis of the mouse genome shows only one gene with a very high homology to the fly Indy gene (NaDC-1). A particularly useful transgenic mouse is one in which the Cre-loxP system is used to disrupt exons 5 through 12 of the mNaDC-1 gene to achieve a functionally null allele of mNaDC-1. This mouse model of the Indy mutation will facilitate the understanding of the role of Indy mutations and caloric restriction in life span extension and serve as a step toward the development of pharmaceutical intervention that may mimic caloric restriction in mammals.

Transgenic animals such as mice, for example, may be used as test substrates for the identification of drugs, pharmaceuticals, therapies and interventions that can be used for the ameliorating or slowing the effects of aging.

Accordingly, the invention features a transgenic organism that contains a transgene encoding a carboxylate transporter polypeptide. The carboxylate transporter polypeptide can transport Krebs Cycle intermediates, such as succinate, citrate, or α-keto-glutarate. The carboxylate transporter polypeptide can function as a cation-independent carboxylate transporter polypeptide. In preferred embodiments, the carboxylate transporter polypeptide is a human carboxylate transporter polypeptide. The carboxylate transporter polypeptide can be exogenous to (i.e., not naturally present in) the transgenic organism.

The transgenic organism can be a yeast cell, an insect, e.g., a worm or a fly, a fish, a reptile, a bird, or a mammal, e.g., a rodent.

The transgenic organism can further comprise a genetic alteration, e.g., a point mutation, insertion, or deficiency, in an endogenous gene. The endogenous gene harboring the genetic alteration can be a gene involved in the regulation of life span, e.g., a gene in the insulin signaling pathway, a gene encoding an carboxylate transport protein, or both. In cases where the genetically altered gene is a carboxylate transporter polypeptide, it is preferable that the expression or activity of the endogenous carboxylate transport protein is reduced or eliminated.

Therapeutic Uses

In another embodiment, the invention features a method of altering the expression or activity of a carboxylate transporter polypeptide, comprising administering to a cell or an organism a compound that increases or decreases the expression or activity of the carboxylate transporter polypeptide in an amount effective to increase or decrease the activity of the carboxylate transporter polypeptide.

Preferably, the carboxylate transporter polypeptide can transport metabolic intermediates, e.g., Krebs Cycle intermediates, e.g., succinate, citrate, or α-keto-glutarate. The carboxylate transporter polypeptide can be a cation-independent carboxylate transporter polypeptide, a human protein, or both. The carboxylate transporter polypeptide can also be a yeast, invertebrate (e.g., worm or fly), or vertebrate (e.g., fish, reptile, bird, or mammal (e.g., mouse)) protein.

The cell to which the compound is administered can be an invertebrate cell, e.g., a worm cell or a fly cell, or a vertebrate cell, e.g., a fish cell (e.g., zebrafish cell), a bird cell (e.g., chicken cell), a reptile cell (e.g., amphibian cell, e.g., Xenopus cell), or a mammalian cell (e.g., mouse or human cell). Similarly, the organism to which the compound is administered can be an invertebrate, e.g., a worm or a fly, or a vertebrate, e.g., a fish (e.g., zebrafish), a bird (e.g., chicken), a reptile (e.g., amphibian, e.g., Xenopus), or a mammal (e.g., rodent or a human). When the organism is a human, it is preferred that the human is not obese or diabetic.

The compound that is administered to the cell or organism can be an agonist that increases the expression or activity of the carboxylate transporter polypeptide or an antagonist that decreases the expression or activity of the carboxylate transporter polypeptide. Whether agonist or antagonist, the compound can be a small organic compound, an antibody, a polypeptide, or a nucleic acid molecule.

The agonist or antagonist can alter the concentration of metabolites, e.g., Krebs Cycle intermediates, e.g., succinate, citrate, or α-keto-glutarate, within the cell or within one or more cells of the organism. Such action is expected to alter the cell's or the organism's resistance to oxidative stress. For example, an antagonist could increase the cell's or the organism's resistance to oxidative stress. In addition, the agonist or antagonist can alter one or more aging-related parameters, e.g., the expression of one or more genes or proteins (e.g., genes or proteins that have an age-related expression pattern), or the value of one or more metabolic parameters (e.g., one or more metabolic parameters that reflect the rate of aging of the cell or organism). the agonist or antagonist alters the rate of aging of the cell or organism.

Ideally, the compound reduces, e.g., partially reduces, the expression of the carboxylate transporter polypeptide. For example, double-stranded inhibitory RNA, anti-sense RNA, or ribozymes can be used to reduce the expression of the carboxylate transporter polypeptide. Double-stranded inhibitory RNA is particularly useful as it can be used to selectively reduce the expression of one allele of a gene and not the other, thereby achieving an approximate 50% reduction in the expression of the carboxylate transporter polypeptide. See Garrus et al. (2001), Cell 107(1):55-65.

In one embodiment, treatment of aging comprises modulating the expression of a carboxylate transporter polypeptide, e.g., an INDY polypeptide. A cell or subject can be treated with an agent that modulates the expression of an Indy gene. These agents can be nucleic acid molecules substantially complementary to an Indy gene. Such approaches include oligonucleotide-based therapies such as antisense, ribozymes, triple helices and double stranded interfering RNAs.

Oligonucleotides may be designed to reduce or inhibit mutant target gene activity. Techniques for the production and use of such molecules are well known to those of ordinary skill in the art. Antisense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Antisense oligonucleotides are preferably 10 to 50 nucleotides in length, and more preferably 15 to 30 nucleotides in length. An antisense compound is an antisense molecule corresponding to the entire Indy mRNA or a fragment thereof.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the well known catalytic sequence responsible for mRNA cleavage disclosed, for example, in U.S. Pat. No. 5,093,246. Within the scope of this disclosure are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites that include the sequences GUA, GUU, and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules used in triple helix formation for the inhibition of transcription should be single stranded and composed of deoxyribonucleotides. The base composition of these oligonucleotides are designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences targeted for triple helix formation may be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Double stranded interfering RNA molecules are also useful; see, for example, Fire et al., Nature 391: 860-11, 1998. Such molecules interfere with the expression of a target gene. For example, double stranded RNA molecules can be injected into a target cell or organism to inhibit expression of a target gene and thus the activity of the gene product. Such double stranded RNA molecules can be more effective at inhibiting gene expression than either strand alone. In addition, double-stranded RNA can be used to specifically target one allele of a gene without effecting the other allele, thus allowing for a partial reduction, e.g., approximately a 50% reduction, in the expression of the gene and corresponding protein. See Garrus et al. (2001), Cell 107(1):55-65.

The antisense, ribozyme, triple helix and/or double stranded interfering RNA molecules described herein may reduce or inhibit the transcription (triple helix) and/or translation (antisense, ribozyme, double stranded interfering RNAs) of mRNA produced by both normal and mutant target gene alleles. If it is desired to retain substantially normal levels of target gene activity, nucleic acid molecules that encode and express target gene polypeptides exhibiting normal activity may be introduced into cells via gene therapy methods that do not contain sequences susceptible to whatever antisense, ribozyme, or triple helix treatments are being utilized. Alternatively, it may be preferable to coadminister normal target gene protein into the cell or tissue in order to maintain the requisite level of cellular or tissue target gene activity.

Antisense RNA and DNA, ribozyme, and triple helix molecules may be prepared by any method known in the art for the synthesis of DNA and RNA molecules. These include techniques for chemically synthesizing oligodeoxyribonucleotides and oligoribonucleotides, for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules may be introduced as a means of increasing intracellular stability and half-life. Possible modifications include but are not limited to the addition of flanking sequences of ribonucleotides or deoxyribonucleotides of the 5′ and/or 3′ ends of the molecule or the use of phosphorothioate or 2′ O-methyl rather than phosphodiesterase linkages within the oligodeoxyribonucleotide backbone.

Modulators of Indy expression are identified in a method wherein a cell is contacted with a candidate compound and the expression of Indy mRNA or protein in the cell is determined. The level of expression of Indy mRNA or protein in the presence of the candidate compound is compared to the level of expression of mRNA or protein in the absence of the candidate compound. The candidate compound can then be identified as a modulator of Indy expression based on this comparison. For example, when expression of Indy mRNA or protein is greater in the presence of the candidate compound than in its absence, the candidate compound is identified as a stimulator of Indy mRNA or protein expression. Alternatively, when expression of Indy mRNA or protein is less in the presence of the candidate compound than in its absence, the candidate compound is identified as an inhibitor of Indy mRNA or protein expression. The level of Indy mRNA or protein expression in the cells can be determined by methods described herein for detecting Indy mRNA or protein. Delivery of antisense, triplex agents, ribozymes, double stranded interfering RNA and the like can be achieved using a recombinant expression vector such as a chimeric virus or a colloidal dispersion system or by injection. Useful virus vectors include adenovirus, herpes virus, vaccinia, and/or RNA virus such as a retrovirus. The retrovirus can be a derivative of a murine or avian retrovirus such as Moloney murine leukemia virus or Rous sarcoma virus. All of these vectors can transfer or incorporate a gene for a selectable marker so that transduced cells can be identified and generated. The specific nucleotide sequences that can be inserted into the retroviral genome to allow target specific delivery of the retroviral vector containing an antisense oligonucleotide can be determined by one of skill in the art.

Another delivery system for polynucleotides is a colloidal dispersion system. Colloidal dispersion systems include macromolecular complexes, nanocapsules, microspheres, beads, and lipid-based systems including oil-in-water emulsions, micelles, mixed micelles and liposomes. A preferred colloidal delivery system is a liposome, an artificial membrane vesicle useful as in vivo or in vitro delivery vehicles. The composition of a liposome is usually a combination of phospholipids, usually in combination with steroids, particularly cholesterol.

The Indy gene may also be underexpressed, causing metabolic or other disorders in particular. Alternatively, the activity of the Indy gene product(s) may be diminished, leading to the development of disease symptoms. Cellular transporter agonists may be used in such cases to increase nutrient uptake for therapeutic reasons in both humans and animals. Therapeutic uses include, but are not limited to, increasing the rate of growth, the rate of weight gain, and the survival rate of premature offspring, neonates, and the aged; increasing total nutrient uptake in subjects with short bowel syndrome or with surgical resection of the intestine; and improving nutritional status of subjects with eating disorders such as anorexia nervosa and bulimia, subjects with acquired immune deficiency syndrome or other chronic immune deficiency syndromes, individuals with Down's syndrome, and burn victims or other severely traumatized subjects.

Methods whereby the level of Indy gene activity may be increased to levels wherein disease symptoms are ameliorated also include increasing the level of gene activity, for example by either increasing the level of Indy gene present or by increasing the level of gene product which is present.

For example, a target gene protein, at a level sufficient to ameliorate metabolic imbalance symptoms, may be administered to a patient exhibiting such symptoms. One of skill in the art will readily know how to determine the concentration of effective, non-toxic doses of the normal target gene protein. Additionally, RNA sequences encoding target gene protein may be directly administered to a patient exhibiting disease symptoms, at a concentration sufficient to produce a level of target gene protein such that the disease symptoms are ameliorated. Administration may be by a method effective to achieve intracellular administration of compounds, such as, for example, liposome administration. The RNA molecules may be produced, for example, by recombinant techniques such as those described above.

Further, patients may be treated by gene replacement therapy. One or more copies of a normal target gene, or a portion of the gene that directs the production of a normal target gene protein with target gene function, may be inserted into cells using

vectors that include, but are not limited to adenovirus, adenoma-associated virus, and retrovirus vectors, in addition to other particles that introduce DNA into cells, such as liposomes. Additionally, techniques such as those described above may be utilized for the introduction of normal target gene sequences into human cells.

Cells, preferably, autologous cells, containing normal target gene expressing gene sequences may then be introduced or reintroduced into the patient at positions which allow for the amelioration of metabolic disease symptoms. Such cell replacement techniques may be preferred, for example, when the target gene product is a secreted, extracellular gene product.

In instances where the target gene protein is extracellular, or is a transmembrane protein, any of the administration techniques described, below which are appropriate for peptide administration may be utilized to effectively administer inhibitory target gene antibodies to their site of action.

The identified compounds that inhibit target gene expression, synthesis and/or activity can be administered to a patient at therapeutically effective doses to treat or ameliorate or delay the symptoms of aging. A therapeutically effective dose refers to that amount of the compound sufficient to result in amelioration or delay of symptoms of aging.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The discovery of the Indy gene provides a therapeutic target for control the rate of aging and the extension of life span. The similarity of INDY to cellular transporters and the localization of INDY suggest that it regulates caloric intake. Unlike other genes previously associated with life-extension in metazoans, Indy appears to be directly involved in intermediary metabolism and thus represents a new class of longevity genes.

While it is presently hypothesized that Indy and its homologs regulate life span and/or caloric intake, all embodiments of this disclosure are equally applicable to any gene product and/or lack of a gene product from the mutations of the Indy gene as described above.

All references cited herein are incorporated by reference in their entirety. The invention is illustrated by the following non-limiting examples.

### EXAMPLES

### Example 1

### Identification of Indy mutants

In order to identify Drosophila strains with extended life spans, Drosophila enhancer-trap lines were studied. An enhancer-trap Drosophila line is one in which a P-element has been inserted. A Drosophila P-element is a transposon that provides a vector for the introduction of a wide variety of genes into the Drosophila germ line. A transposon is a DNA element that promotes its own transposition between different genetic loci. A wild-type Drosophila P-element is 2.9 kb in length and can include the gene for transposase, an enzyme that facilitates transposition. The enhancer-trap P-element cannot mobilize itself, but requires the presence of transposase from another source. The enhancer-trap P-element is a modified P-element which is about 10 kb and contains such DNA sequences as the P-element long terminal like repeats, the gene for bacterial lacZ, the white minigene which gives the fly a pigmented eye, a region for plasmid rescue, an origin of replication and ampicillin resistance. The P-element also contains a minimal promoter region, which is insufficient itself to induce transcription, but can be transcribed if inserted in a region of the Drosophila genome that contains an enhancer. Because the P-element contains the lacZ gene, the activity of the reporter gene can be assayed using standard β-galactosidase assays. The P-elements thus insert somewhat randomly into the Drosophila germ line, and may interfere with gene expression and essentially become mutants that can be assayed for a particular phenotype. The Drosophila enhancer lines used are described in Boynton and Tully, Genetics 131: 655-72, 1992.

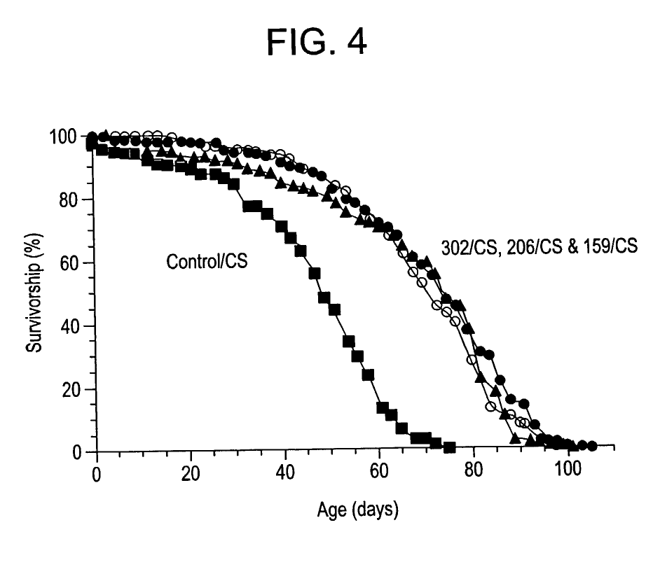

In the search for long-lived Drosophila mutants, it was observed that two fly lines, 206 and 302, showed a near doubling of mean life-span (from 37 to 70 days) and a 50% increase in maximal life-span (FIG. 4). The mean 25° C. life spans of controls were 37 days, while the mean life spans for Indy206, Indy302, and Indy159 were 71, 69, and 69 days respectively. Indy206, Indy302, and Indy159 extended mean life span by 92%, 87%, and 87% respectively. Extension of 1% maximal life span of these Indy mutants was greater than 45%. At 18° C., the increase in mean life span conferred by Indy mutations approaches 100% while the increase in 1% maximum life span approaches 50%. Flies were maintained in a humidified, temperature controlled environmental chamber at 25° C., transferred to fresh food vials and scored for survival every 2 to 3 days. Each survivorship curve represents data from over 300 male flies. A total of 5430 male and female Indy heterozygote flies were tested.

The increase in life span occurred only in heterozygotes, that is flies with only one copy of the enhancer-trap chromosome and a copy of a normal Indy gene. Chromosomal in situ hybridization revealed that the P-element in both the 206 and 302 cell lines was inserted at the same cytological location. Genomic DNA flanking the site of insertion in the 206 and 302 cell lines was obtained by plasmid rescue and sequenced. The insertion sites in the 206 and 302 enhancer trap cell lines were 5753 base pairs from each other and were in the same gene, which has been named Indy (for I'm not dead yet). The Indy cDNA is SEQ ID NO:1.

### Example 2

### Identification of the Indy Gene

Information on the chromosomal location of Indy was used to identify additional mutations in the Indy gene from other laboratories. Several candidate lines with P-element insertions in the same cytogenic location as Indy were examined. A third enhancer-trap line, the 159 fly line, with a P-element inserted 734 base pairs from the site of the 206 insertion was identified. As a heterozygote, this 159 fly line showed a similar extension in life span to the other Indy insertions (FIG. 4). Two further P-element insertions in Indy were obtained through site-selected mutagenesis of the Indy locus. In a polymerase chain reaction-based screen of 10,000 mutagenized third chromosomes, two new insertions into the Indy locus, lines 92 and 265, were identified. Flies heterozygous for either the 92 or 265 allele showed extension of life span similar to that of the original selected mutants. The accession number for the Indy cDNA is AF217399 and the FlyBase number is CG3979.

The genomic organization of the Indy locus with the insertion sites of all five P-element alleles is shown in FIG. 3. The organization of the Indy transcription unit is shown, with the “ATG” initiation codon and “TAG” stop codon noted. Solid black boxes represent the conserved AntC and Fas1 nucleotide sequences found 5′ of the transcriptional start site of the Indy gene and that are thought to be involved in regulation of gene expression. AntC has a high level of homology to the 5′ region of the Antennapedia gene. Fas1 has a high level of sequence homology to the 5′ region of the Fasiculin 1 gene. The gray rectangle is the sequence of the conserved Hoppel transposable element found in the first intron of the Indy gene. Hoppel is another type of transposable element in Drosophila and this element is found in the first intron of the Indy gene is wild type flies. The original enhancer-trap lines are shown as PlacW302, PlacW206 and PlacW159. The orientation of the insertion is indicated 5′ to 3′ by the black arrow. Two of the original lines are insertions in the Hoppel element in the first intron just upstream of the coding region (206 and 159). The third original Indy stock (302) has its insertion just upstream of the transcriptional start site. The Indy mutants generated by site-selected mutagenesis are indicated as Birmingham-2 P-element insertions PBm92 and PBm265. The PlacW insertion is not drawn to scale.

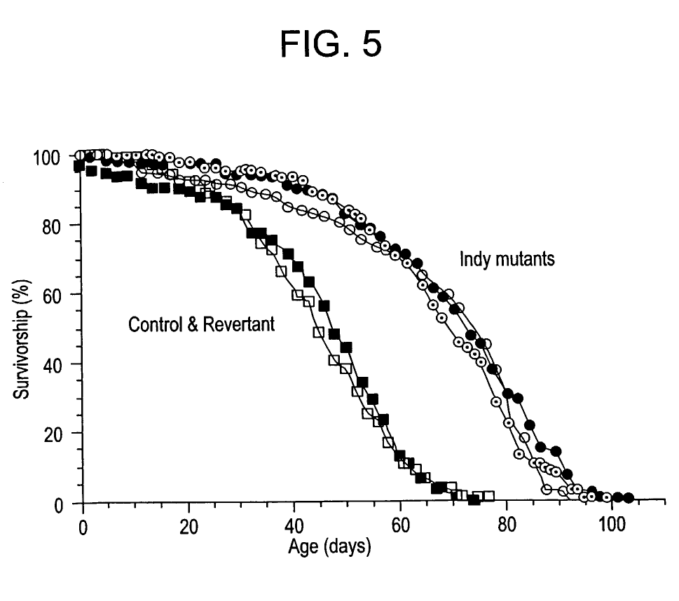

To confirm that the P-element insertion in Indy caused the observed life-span extension, the P-element was remobilized and excised from the 206 and 302 lines. Four independent lines of flies, shown by sequence analysis to carry exact excisions, reverted to normal lifespan (FIG. 5). A nonexcision control line isolated at the same time which passed through the same genetic background as the excision lines remained long-lived.

### Example 3

### Confirmation that the Indy Mutations have a Positive Effect on Life Span

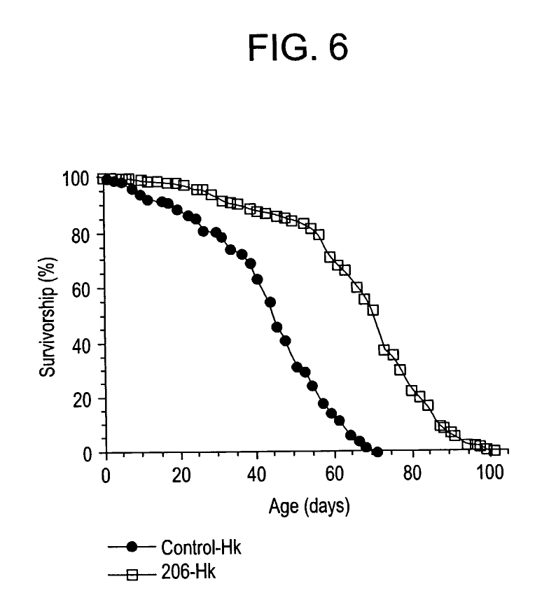

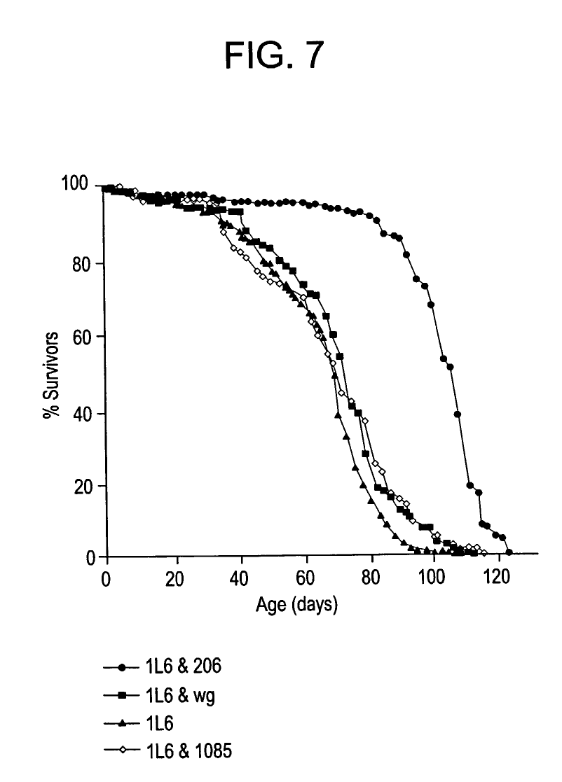

To exclude the possibility that the extended life span of the Indy mutants was due to the rescue of uncharacterized deleterious mutations accumulating in the wild-type Canton-S stock, the Indy mutation was crossed into several different genetic backgrounds distinct from the Canton-S stock. The stocks tested included the Hyperkinetic, Shaker, and drop dead stocks, each of which was isolated from other laboratory stocks over 25 to 30 years ago. Also tested was a long-lived Luckinbill laboratory selected line. In all cases, there was an extension of life span. For Hyperkinetic, Shaker and drop-dead, the mean life span was extended 40-80% (FIG. 6). For the Luckinbill line, life span was additionally extended by 15% (FIG. 7). These data indicate that the mechanism by which Indy mutations extend life span is a positive effect of the mutation and not simply the effect of rescuing deleterious mutations. The smaller increase in life-span associated with the laboratory-selected long-lived lines provides additional evidence that the mechanisms by which Indy acts to increase life-span may represent physiological systems already partially optimized by laboratory selection.

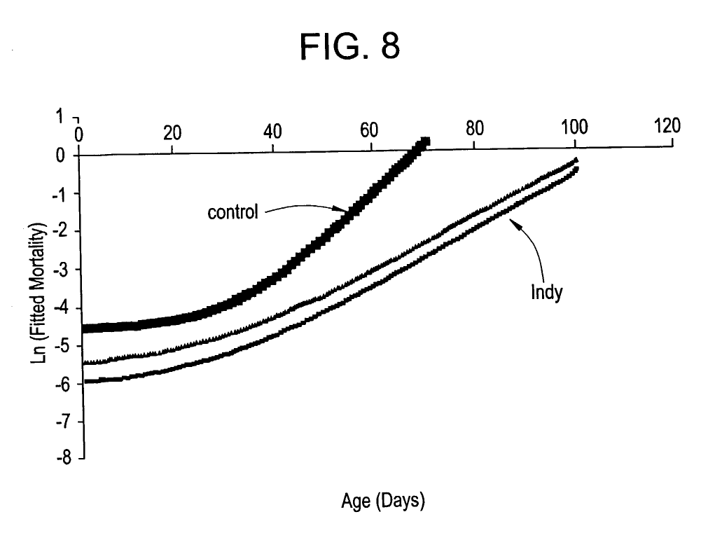

Life span can be increased with or without a change in the rate of aging. Treatments such as lowering growth temperature and caloric restriction decrease the rate of aging. In most long-lived Drosophila mutants that have been characterized, the aging process is simply delayed without a change in the rate of aging. Indy heterozygous mutants, however, show a significant decrease in the rate of aging (FIG. 8). Indy is thus the first long-lived Drosophila mutant to show a change in the rate of aging rather than simply a delay in the initiation of the aging process.

### Example 4

### Characterization of the Indy Gene Product

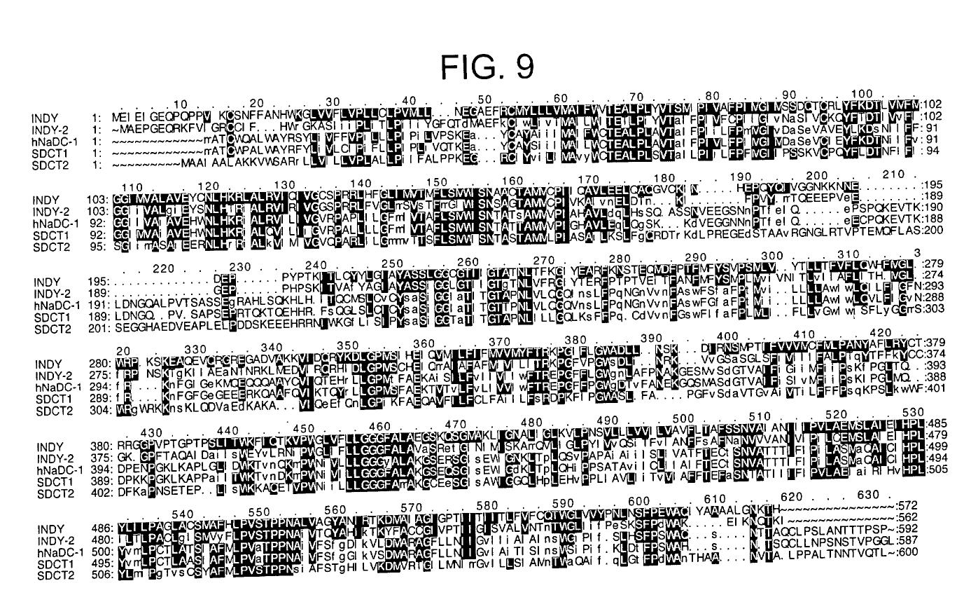

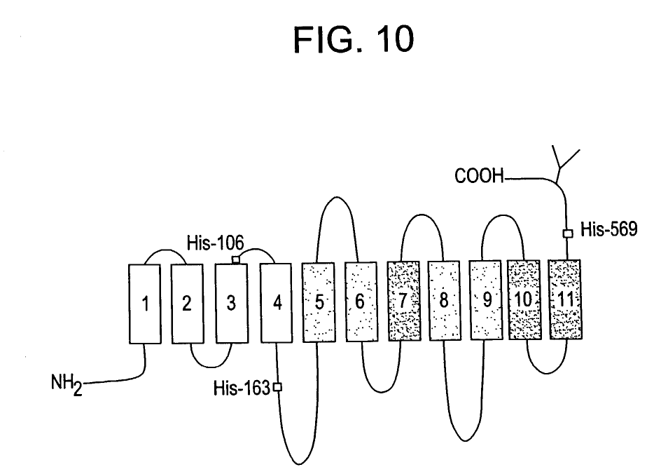

Genomic and cDNA sequences of the Indy gene predicted a 572-amino acid protein (Seq. ID NO. 2) with 34% identity and 50% similarity to human, mouse and rat renal sodium dicarboxylate cotransporters (FIG. 9). The accession number for the Indy polypeptide is AE003519. Mammalian dicarboxylate cotransporters are membrane proteins responsible for the uptake or re-uptake of di- and tricarboxylic acid Krebs cycle intermediates such as succinate, citrate, and alpha-ketoglutarate. A schematic of a dicarboxylate cotransporter is shown in FIG. 10. They are found in a variety of tissues, including brush border cells of the small intestine, colon and placenta; the basolateral membrane of perivenous cells in the liver; and epithelial cells of the renal proximal tubule and the brain. Dicarboxylate cotransporters are also found in the placenta and brain of mammals.

### Example 5

### Localization of the Indy Gene Product

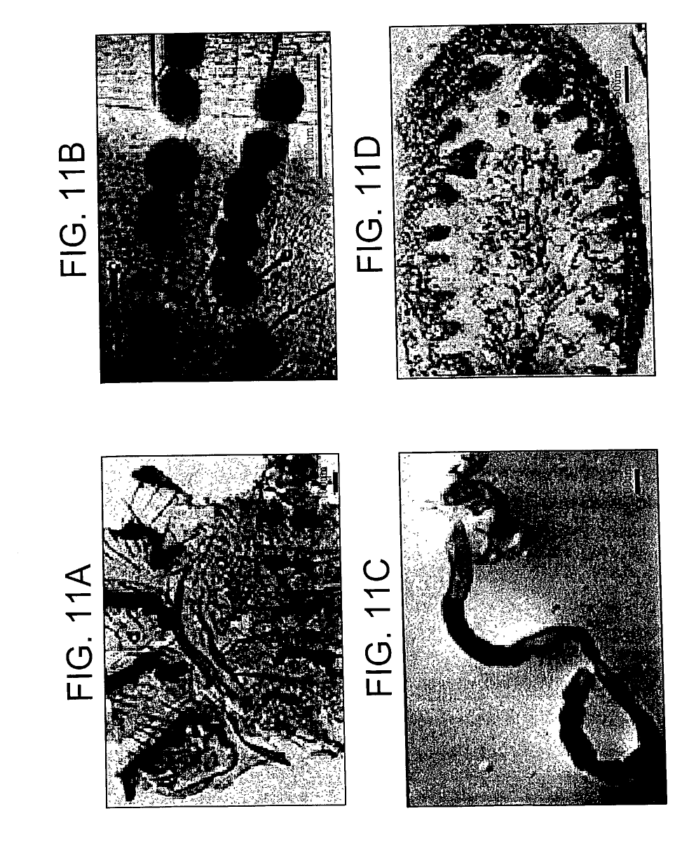

The P-element insertion encodes a reporter protein $-galactosidase ($-gal), which allows localization of the Indy gene message. Expression of $-gal was visualized by X-gal staining. Indy has an identical pattern of expression in the 206, 302 and 159 enhancer-trap lines despite the P-elements being almost 6.5 kb away from each other in the three lines. In adult flies, Indy is expressed in the fat body, midgut, and oenocytes (FIG. 11). These organs are thought to be the primary sites of intermediary metabolism, absorption, and metabolic storage in Drosophila. The fat body is involved in the metabolism and storage of fat, glycogen, and protein and is most often compared to the liver in vertebrates. Indy was also expressed at lower levels in the halteres; portions of the alimentary canals, including the procardia and restricted regions of the esophagus and hindgut; and the base of the legs. These are regions that have been identified as storage deposits for glycogen. Finally, Indy was expressed in a subset of cells in the third segment of the antennae.

The localization of INDY was also determined using staining with an anti-INDY antibody. Two INDY peptides were used to generate antibodies in a rabbit: amino acid residues 181-197 of SEQ ID NO:2 (EPQYQIVGGNKKNNEDE (SEQ ID NO:7)) and amino acid residues 281-298 of SEQ ID NO:2 (RPKSKEAQEVQRGREGADVA (SEQ ID NO:8)).

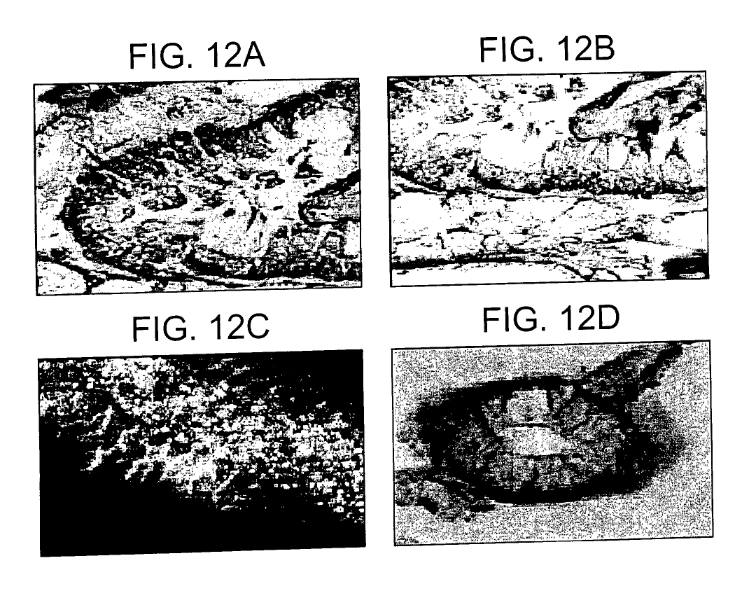

The synthesis of an immunogenic peptide is followed by injection into two New Zealand white rabbits. Subsequent boosts and bleeds are taken according to our ten-week protocol. We received 5 mgs of peptide, aliquots of prebleeds., roughly 80 ml of crude sera from each of the two rabbits, and ELISA titration data. INDY staining was visualized by using an anti-rabbit secondary antibody coupled to horseradish peroxidase (HRP) and then reacted with DAB to visualize INDY staining as a brown precipitate (FIG. 12). The use of the antibodies demonstrated that the location of the INDY protein is primarily the plasma membrane of the cells in which it is expressed. More specifically, in the midgut, INDY is expressed prominently on the basolateral portion of the epithelium, and possibly the apical region.

### Example 6

### Biological Function of Indy

To confirm that INDY is a transporter, the Indy mRNA was injected into Xenopus laevis oocytes. Stage V and VI oocytes from Xenopus laevis were dissected and defoliculated. The oocytes were injected with 25-50 ng of Indy mRNA 1 day after isolation. Nutrient uptake was measured 6 to 7 days later. The oocytes were maintained at 18° C. in Barth's solution containing 50 mg/ml gentamycin sulfate, 2.5 mM sodium pyruvate, and 5% heat-inactivated horse serum (Coady et al., Arch. Biochem. Biophys. 283: 130-134, 1990; Pajor and Wright, J. Biol. Chem 26:, 3557-3560, 1992).

Transport measurements were performed as in Pajor, J. Biol. Chem. 270: 5779-5785, 1995. Groups of oocytes were first washed in choline buffer to remove the serum. Transport was initiated by adding 0.4 ml of [¹⁴C]succinate or [14C] citrate in buffer A. Buffer A comprises 100 mM NaCl, 2 mM KCl, 1 mM MgCl₂, and 10 mM HEPES-Tris (pH 7.5). Buffer B is the same as buffer A except the NaCl is replaced by 100 mM choline Cl. For other cation replacement studies, the sodium was replaced by equimolar amounts of the other cations, as their chloride salts. At the completion of the transport time, the nutrient uptake was stopped with 4 washes of 4 ml of ice-cold buffer B. To count the amount of [¹⁴C] taken up by the oocyte, each oocyte was dissolved in 0.5 ml of 10% SDS, and the [¹⁴C] assayed by scintillation counting.

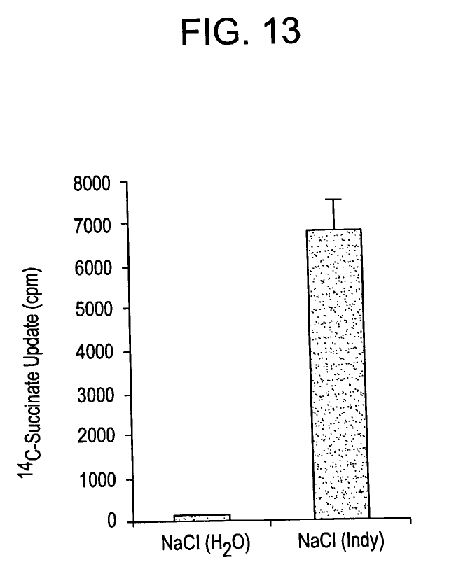

When Indy mRNA was injected into Xenopus oocytes, greater than a 100-fold increase was observed in the uptake of [¹⁴C] succinate as compared to a control H₂O injected oocyte (FIG. 13). This increase in succinate transport is comparable to that observed for sodium dicarboxylate cotransporters such as the rat renal transporter.

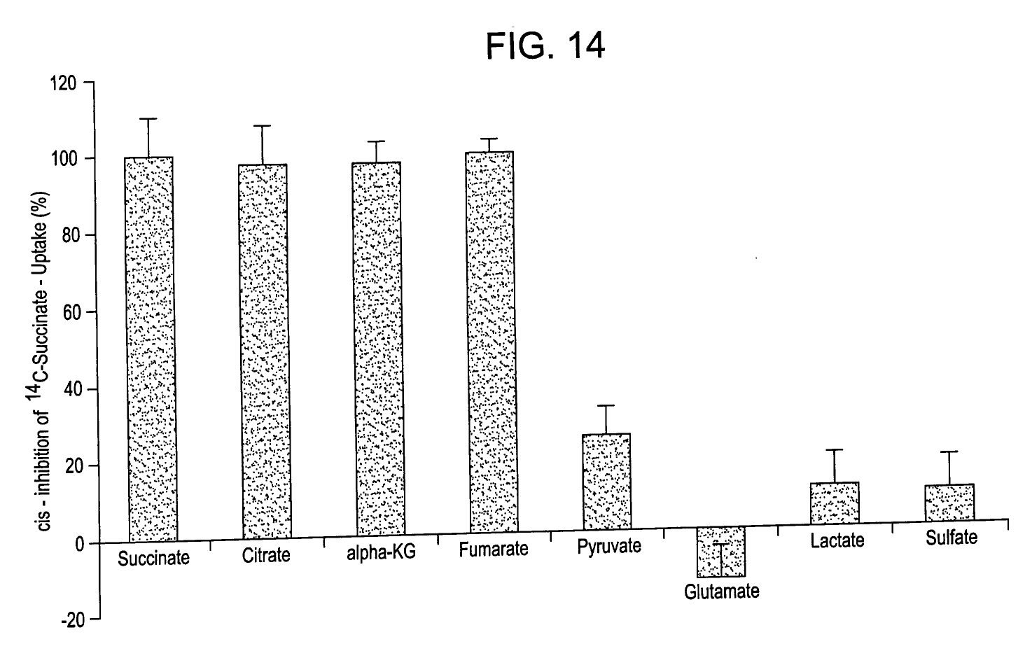

Studies of sodium dicarboxylate cotransporters have indicated a broad range of specificity for di- and tri-carboxylic acids and the exclusion of monocarboxylic acids. The transport specificity of Indy was thus determined as the ability of various compounds to inhibit the uptake of [¹⁴C]succinate. The test inhibitors were added at a concentration of 1 mM, approximately a 100-fold excess of inhibitor. As seen in FIG. 14, succinate, citrate, alpha-ketoglutarate, and fumarate inhibit succinate uptake nearly 100%. There is little inhibition of succinate transport by pyruvate, glutamate, lactate, or sulfate.

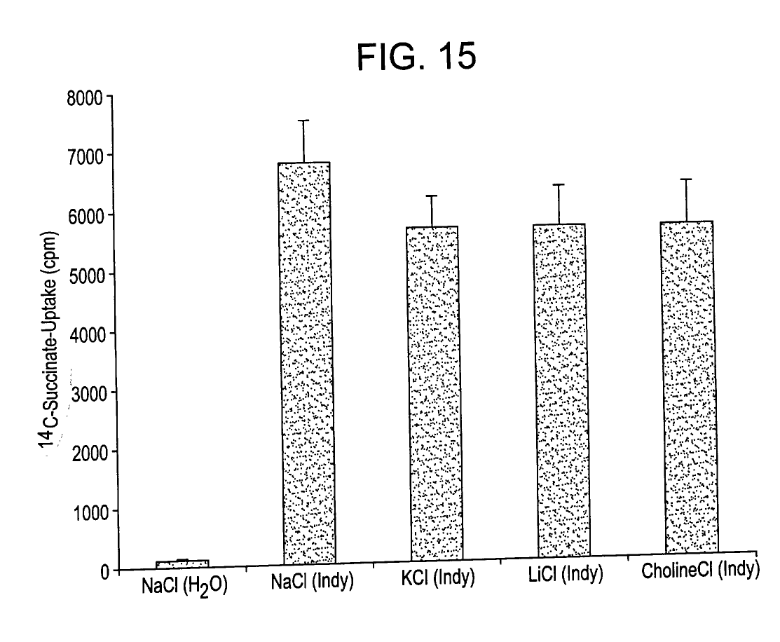

One feature of the sodium dicarboxylate cotransporters that has been studied is cation-dependence of the transport. Transport of succinate in the rabbit renal sodium dicarboxylate cotransporter is dependent on a cation, preferably sodium (Pajor et al., J. Biol. Chem. 273: 18923-18929, 1998). Lithium can support transport in the absence of sodium, but becomes an inhibitor in the presence of sodium. It is speculated that lithium has a high affinity for one of the three postulated sodium binding sites in the cotransporter. In contrast to lithium, choline is a potent inhibitor of succinate transport suggesting that the rabbit renal dicarboxylate cotransporter is a true transporter requiring cation cotransport. The cation specificity of Indy was thus examined. In contrast to the sodium dicarboxylate cotransporters, Indy demonstrates similar transport in the presence of sodium, lithium, potassium, and choline (FIG. 15). This result strongly suggests that Indy is a transporter rather than a cotransporter. Thus Indy may represent a new class of cation-independent dicarboxylate transporters.

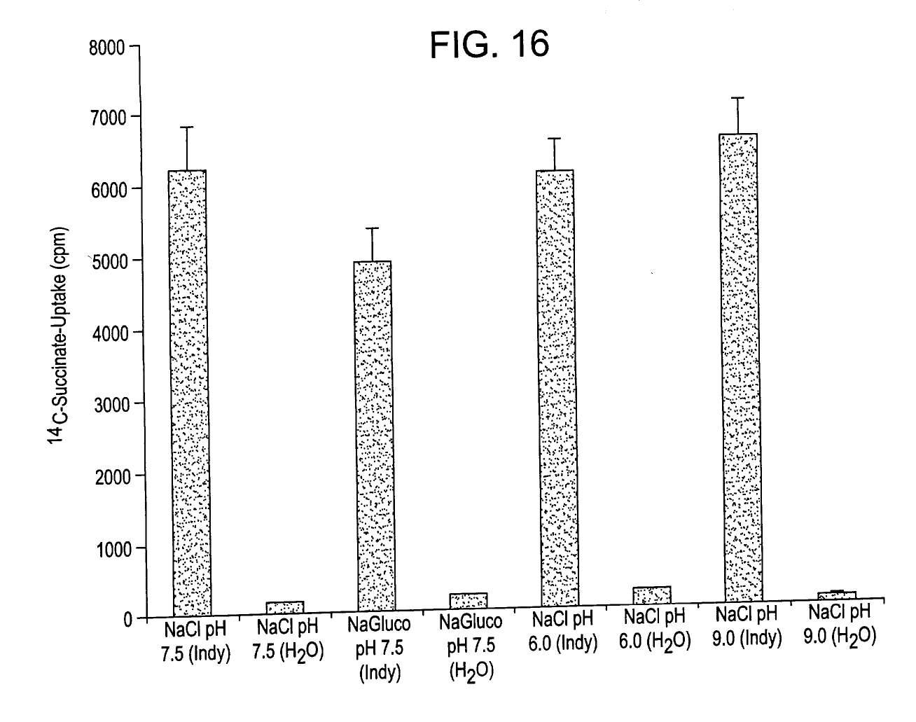

The pH-dependence of succinate transport was also determined. As with the sodium dicarboxylate cotransporters, succinate transport by INDY shows very little dependence on pH and appears not to be using protons as a cotransporter (FIG. 16).

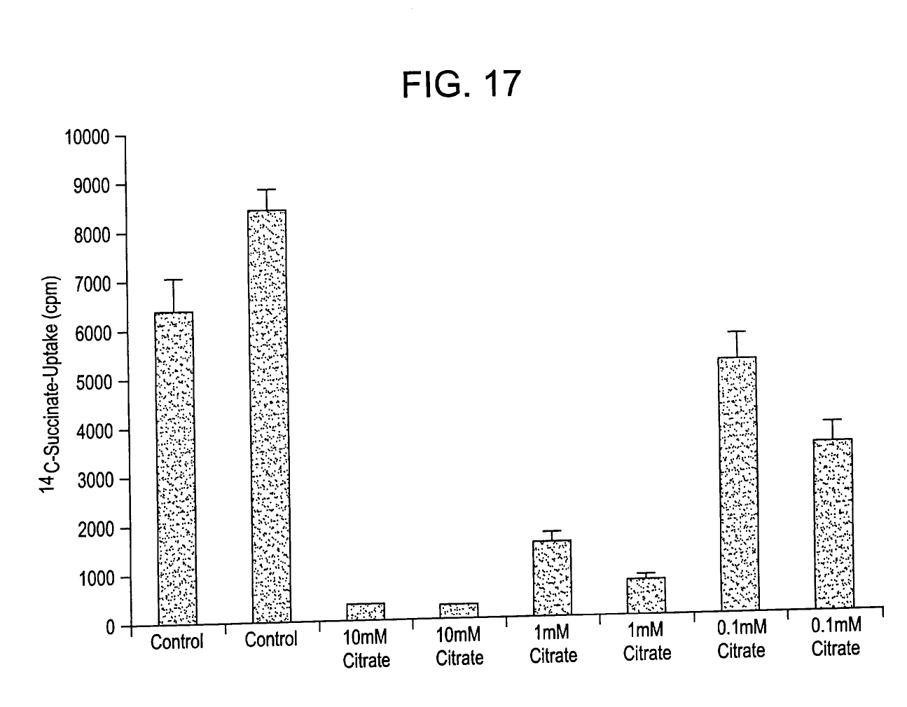

The relative affinity of INDY for succinate and citrate was determined by adding a constant amount of [¹⁴C]succinate and increasing concentrations of citrate to oocytes expressing INDY. As seen in FIG. 17, citrate at 10 mM completely inhibits succinate uptake, citrate at 1 mM inhibits succinate uptake by about 90% and 0.1 mM citrate inhibits succinate uptake by about 50%.

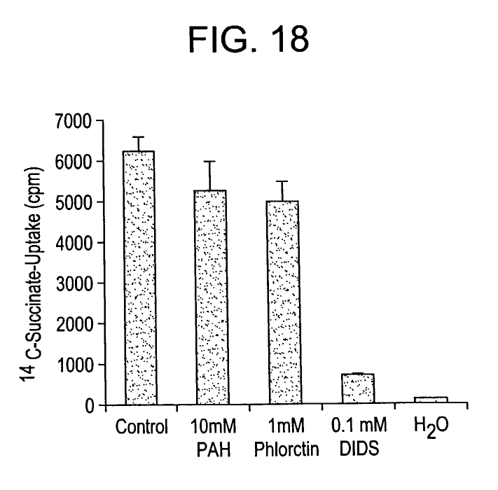

To further characterize INDY, inhibitors of transport were added to the Indy oocyte system. P-aminohippuric Acid (PAH) is a reference compound used to study transport mechanisms. 4,4′-Diisothyocyanostilbene-2,2′-disulfonic acid (DIDS) is a specific inhibitor of channels. Phloretin is an inhibitor of channels that is known to block protein kinase C. Both PAH and phloretin have no effect on succinate transport while DIDS inhibits succinate transport by 80% (FIG. 18). The lack of inhibition by phloretin is interesting because phloretin has been shown to inhibit both inward and outward currents in sodium-dependent transport in the rat sodium dicarboxylate cotransporter (Chen et al., J. Biol. Chem. 273:20972-20981, 1998. Because phloretin does not inhibit INDY, this is further indication that INDY represents a new class of dicarboxylate transporters.

### Example 7

### Effect of Indy on Fertility and Physical Activity

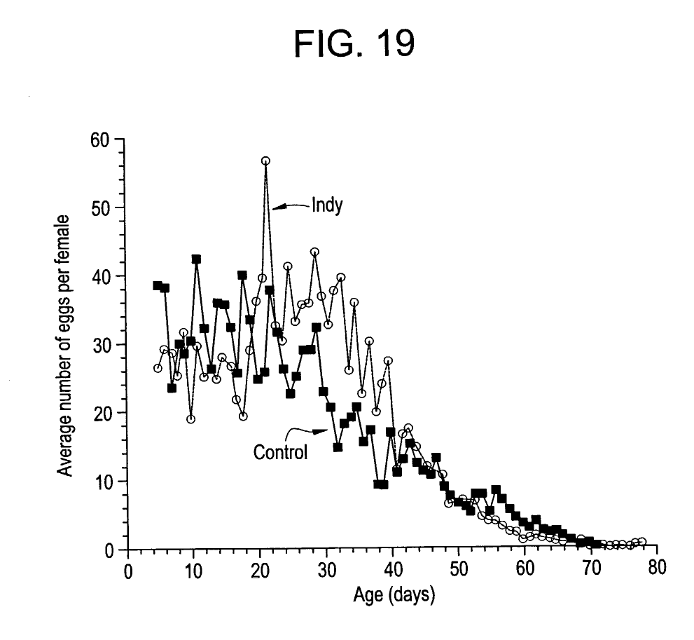

A decline in fertility or a reduction in physical activity can lead to an extension of life span in flies. Indy long-lived heterozygote males and females were compared to controls and found to be normal or superior in fertility and fecundity. Female fertility as measured by egg production is shown in FIG. 19. Qualitative observations of flight, courtship, feeding behavior and negative geotaxis revealed no significant differences between Indy long-lived males and females during early life. Differences occurred later in life when physical measures of behavior and locomotor function were maintained at high levels in Indy long-lived animals but not in normal-lived controls. For instance, one physiological milestone of aging in flies is the onset of female infertility. Indy heterozygous long-lived females continued to produce viable adult offspring 40% longer on average than did control flies (23.2 vs. 16.5 days). This was a true extension of the period of fertility and was not associated with a compensatory delay in fertility during early life, as seen in laboratory-selected long-lived lines. Indy long-lived females showed the same early peak of egg laying and fertility as control females but sustained the ability to produce larger numbers of offspring for a longer period of time. There was no alteration in the rate or timing of developmental events in Indy long-lived mutant animals, as in the C. elegans clock mutants. The time from egg to adult at 25° was the same as for normal controls (9 to 10 days). Studies on metabolic rate have also showed that Indy long-lived mutants have the same metabolic rate as controls suggesting that the increase in life-span in Indy mutants is not due to a slowing down of metabolic rate.

### Example 8

### Relationship of Indy to Caloric Restriction

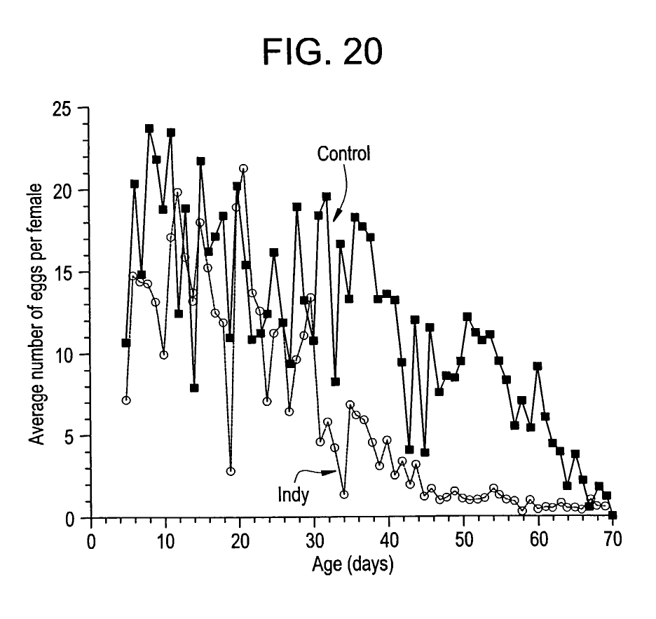

It has been proposed that Indy acts through a caloric restriction mechanism. Under normal high calorie feeding conditions, the egg production in Indy heterozygous females is comparable to normal females (FIG. 19). Under low calorie conditions, however, the egg production in Indy heterozygous females is significantly lower than that in normal females (FIG. 20). This result strongly suggests that Indy females are already calorically restricted and that by further restricting them through diet, a deleterious effect on fertility is observed.

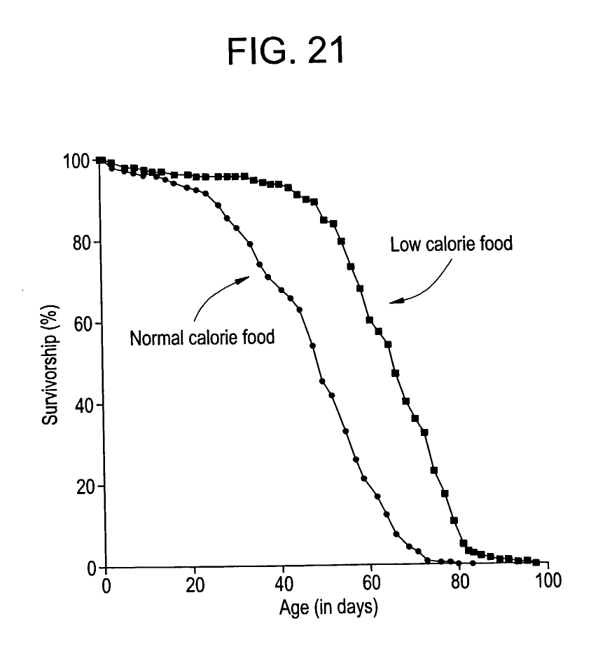

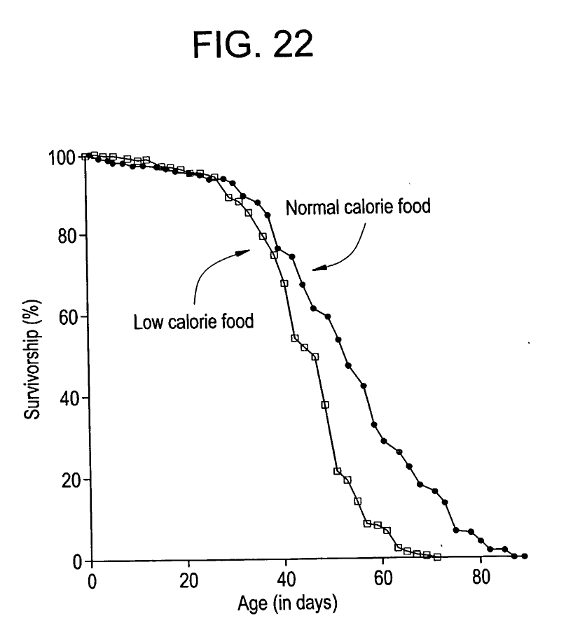

Further evidence for the relationship between Indy long-lived effects and caloric restriction is seen in the effect of caloric restriction on Indy mutant flies. In a normal fly, caloric restriction increases life-span (FIG. 21). In an Indy/Indy mutant homozygous fly, caloric restriction decreases life-span (FIG. 22). This result suggests that further restriction of calories (such as through diet) in an Indy homozygous mutant fly decreases life span. It is thus believed that Indy mutants act by calorically restricting the flies and in heterozygotes (Indy/normal) flies the level of caloric restriction results in an increase in life-span.

### Example 9

### Expression of Indy in Yeast

The Indy cDNA was ligated into pRS426-Gal using standard molecular biology techniques. The yeast cells were then transformed with the Indy-pRS426-Gal plasmid using standard protocols.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A method of evaluating a compound, the method comprising:
contacting a transporter polypeptide with a test compound;
evaluating an interaction between the test compound and the transporter polypeptide;
contacting a cell or organism that produces the transporter polypeptide with the test compound; and
evaluating rate of aging of the cell or organism.

2. The method of claim 1, wherein evaluating the rate of aging comprises one or more of:
(a) assessing the life span of the cell or the organism;
(b) assessing the presence or abundance of a gene transcript or gene product in the cell or organism that has a biological age-dependent expression pattern;
(c) evaluating resistance of the cell or organism to stress,;
(d) evaluating one or more metabolic parameters of the cell or organism;
(e) evaluating the proliferative capacity of the cell or a set of cells present in the organism; and
(f) evaluating physical appearance or behavior of the cell or organism.

3. The method of claim 1, wherein the transporter polypeptide is carboxylate transporter polypeptide.

4. The method of claim 3, wherein evaluating the interaction between the test compound and the transporter polypeptide comprises evaluating binding of the test compound to the transporter polypeptide.

5. The method of claim 3, wherein evaluating the interaction between the test compound and the transporter polypeptide comprises evaluating a biological activity of the transporter polypeptide.

6. The method of claim 3, wherein an organism is contacted with the test compound and the effect of the test compound on the rate of aging of the organism is evaluated.

7. The method of claim 6, wherein the organism is a nematode, a fly, or a rodent.

8. The method of claim 6, wherein the organism is a transgenic organism.

9. The method of claim 8, wherein the transgenic organism comprises a transgene encoding a heterologous transporter polypeptide.

10. The method of claim 8, wherein the transgenic organism comprises a transgene that encodes a human carboxylate transporter polypeptide.

11. The method of claim 6, wherein at least 60% of the expected normal life span of the organism has elapsed prior to the organism being contacted with the test compound.

12. The method of claim 6, wherein evaluating the rate of aging comprises measuring the life span of one or more organisms contacted with the test compound.

13. The method of claim 12, wherein a statistical value descriptive of life span for a group of genetically matched organisms contacted with the test compound is measured and that statistical value is compared to an equivalent statistical value descriptive of life span for a control group of genetically matched organisms that have not been contacted with the test compound.

14. The method of claim 13, wherein the statistical value comprises an average or a median.

15. The method of claim 3, wherein the rate of aging of the cell is determined.

16. The method of claim 15, wherein the cell is isolated from an organism that has been contacted with the test compound.

17. The method of claim 15, wherein the cell is contacted with the test compound in vitro.

18. The method of claim 15, wherein the cell is a transgenic cell.

19. The method of claim 18, wherein the transgenic cell comprises a transgene encoding a heterologous carboxylate transporter polypeptide.

20. The method of claim 18, wherein the transgenic cell comprises a transgene encoding a human carboxylate transporter polypeptide.

21. The method of claim 3 further comprising repeating the method with one or more additional compounds to thereby evaluate a library of test compounds.

22. The method of claim 3, wherein the carboxylate transporter polypeptide comprises the amino acid sequence of SEQ ID NO:2.

23. The method of claim 3, wherein the carboxylate transporter polypeptide comprises a mammalian amino acid sequence.

24. The method of claim 23, wherein the carboxylate transporter polypeptide comprises a human amino acid sequence.

25. A method of evaluating a compound, the method comprising:
providing a cell or organism, wherein the cell or organism expresses a heterologous transporter polypeptide;
administering a test compound to the organism; and
evaluating an age-associated parameter of the cell or organism.

26. The method of claim 25, wherein the method provides, administers to, and evaluates the organism.

27. The method of claim 26, wherein the organism is a nematode, a fly, or a rodent.

28. The method of claim 27, wherein the organism is a fly.

29. The method of claim 28, wherein the fly comprises a transgene encoding a carboxylate transporter polypeptide.

30. The method of claim 28, wherein the fly comprises a transgene encoding a human transporter polypeptide.

31. The method of claim 28, wherein the fly is deficient in an endogenous carboxylate transport activity.

32. The method of claim 25, wherein the method provides, administers to, and evaluates a cultured cell.

33. The method of claim 32, wherein the cultured cell is a mammalian cell.

34. A method of evaluating a protein, the method comprising:
a) identifying a candidate protein, wherein the candidate protein is a transporter polypeptide;
b) altering the expression or activity of the candidate protein in a cell or in one or more cells of an organism; and
c) evaluating the rate of aging of the cell or the organism, thereby evaluating the protein.

35. The method of claim 34, wherein an organism is used to evaluate the protein.

36. The method of claim 35, wherein the transporter polypeptide is a carboxylate transporter polypeptide.

37. The method of claim 34, wherein the alteration is a reduction in the expression of the candidate protein.

38. The method of claim 37, wherein the expression of the candidate protein is reduced using double stranded inhibitory RNA, antisense RNA, or a ribozyme.

39. The method of claim 34, wherein the alteration is an increase in the expression of the candidate protein.

40. The method of claim 39, wherein the increase is produced by a transgene that encodes a copy of the candidate protein.

41. A method of evaluating a protein, the method comprising
a) identifying a candidate protein, wherein the candidate protein is a transporter polypeptide;
b) identifying one or more polymorphisms in the gene that encodes the candidate protein; and
c) determining whether there is a correlation between the presence of one or more of the polymorphisms and the longevity of the organism that contains the polymorphism,
thereby evaluating the protein.

42. A method of altering the rate of aging of a cell or organism, comprising administering to a cell or an organism a compound that alters the expression or activity of a transporter polypeptide in an amount effective to alter the activity of the transporter polypeptide, wherein the transporter polypeptide comprises at least eleven transmembrane domains and can transport a dicarboxylate, a tricarboxylate, or sulfate.

43. The method of claim 42, wherein the carboxylate transporter polypeptide is a cation independent carboxylate transporter polypeptide.

44. The method of claim 42, wherein the compound is a small organic molecule, a peptide, an antibody, or a nucleic acid molecule.

44. The method of claim 43, wherein the compound is a double-stranded inhibitory RNA molecule.

45. The method of claim 44, wherein the transporter polypeptide is at least 25% identical to SEQ ID NO:2.

46. The method of claim 40, wherein the rate of aging of an organism is altered.

47. The method of claim 46, wherein the organism is a mammal.

48. The method of claim 47, wherein the organism is a human.

49. The method of claim 48, wherein the human is not obese or diabetic.

50. A transgenic Drosophila that produces a mammalian transporter polypeptide in one or more cells, wherein the mammalian transporter polypeptide comprises at least eleven transmembrane domains and can transport a dicarboxylate, a tricarboxylate, or sulfate.

51. The Drosophila of claim 50, wherein the Drosophila is deficient in an endogenous carboxylate transporter activity.

52. The Drosophila of claim 51, wherein the activity is the activity of Drosophila Indy.

53. The Drosophila of claim 52, wherein the Drosophila comprises a defective allele of the Indy gene.

54. The Drosophila of claim 53, wherein the Drosophila is heterozygous for the Indy gene.

55. The Drosophila of claim 50, wherein the mammalian carboxylate transporter is produced at least in cells of the gut.

56. The Drosophila of claim 50, wherein a nucleic acid encoding the mammalian carboxylate transporter is operably linked to a transcriptional regulatory sequence of the Indy gene.

57. A method comprising:
contacting the Drosophila of claim 50 with a test compound; and
evaluating a phenotype of the Drosophila.

58. The method of claim 57, wherein the phenotype being evaluated is the rate of aging.

59. The method of claim 3 wherein the transporter polypeptide is at least 25% identical to SEQ ID NO:2.

60. The method of claim 1 wherein the transporter polypeptide is at least 55% identical to SEQ ID NO:3, 4, 5, or 7.

61. The method of claim 1 wherein the transporter polypeptide can transport a sulfate ion.

62. The method of claim 61 wherein the transporter polypeptide comprises at least eleven transmembrane domains.

63. The method of claim 61 wherein the transporter polypeptide is human.

64. The method of claim 60 wherein the transporter polypeptide comprises the amino acid sequence of SEQ ID NO:3, 4, 5, or 7.
